(19) United States
(12) Patent Application Publication
(10) Pub. No.: US 2026/0253732 A1
(43) Pub. Date: Aug. 27, 2026

(54) SYSTEM

(71) Applicant: SoftBank Group Corp., Tokyo (JP)

(72) Inventor: Yota KASHIMA, Tokyo (JP)

(73) Assignee: SoftBank Group Corp., Tokyo (JP)

(21) Appl. No.: 19/541,413

(22) Filed: Feb. 17, 2026

(30) Foreign Application Priority Data
Feb. 21, 2025 (JP) 2025-026972

(51) Int. Cl. G16H 50/20; G06F 16/9535

(52) U.S. Cl. CPC G16H 50/20; G06F 16/9535

(57) ABSTRACT

The system according to the embodiment comprises a reception unit, an analysis unit, a provision unit, and a dissemination unit. The reception unit is configured to receive as input a small number of parameters of a user's basic information or past medical history. The analysis unit analyzes the information received by the reception unit and supplements information necessary for diagnosis using unique information of each individual as a key. The provision unit provides a treatment method or advice based on a diagnosis result predicted by the analysis unit. The dissemination unit sends a message prompting the child generation to use a similar service when the parent generation receives online medical care.

### CROSS-REFERENCE TO RELATED APPLICATION

The present application claims priority to and incorporates by reference the entire contents of Japanese Patent Application No. 2025-026972 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there have been issues such as insufficient information necessary for diagnosis in online medical care and difficulties in disseminating such services to the elderly generation.

### SUMMARY OF THE INVENTION

The system according to the embodiment comprises a reception unit, an analysis unit, a provision unit, and a dissemination unit. The reception unit is configured to receive as input a small number of parameters of a user's basic information or past medical history. The analysis unit analyzes the information received by the reception unit and supplements information necessary for diagnosis using unique information of each individual as a key. The provision unit provides a treatment method or advice based on a diagnosis result predicted by the analysis unit. The dissemination unit sends a message prompting the child generation to use a similar service when the parent generation receives online medical care.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

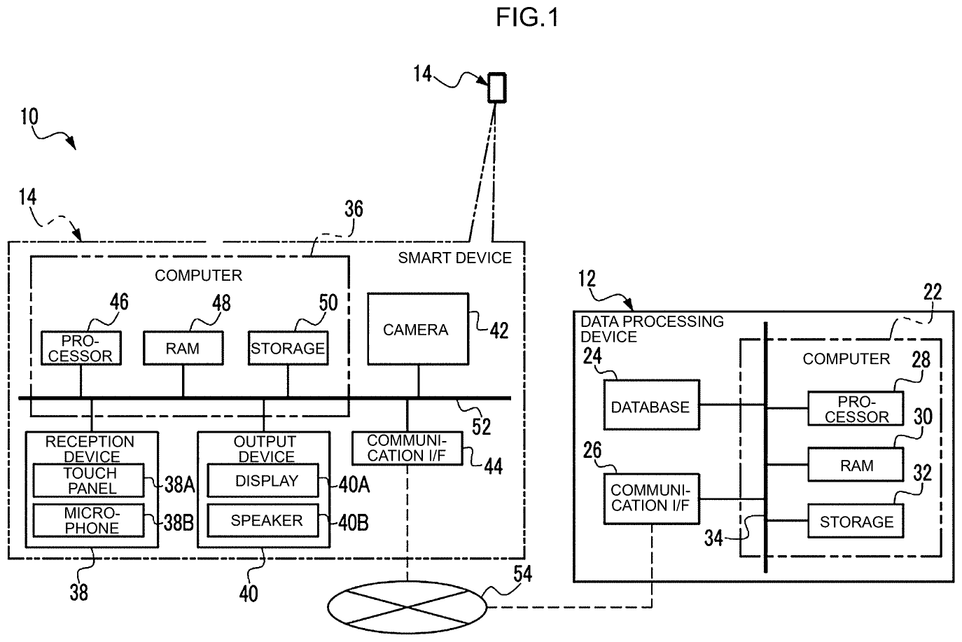

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

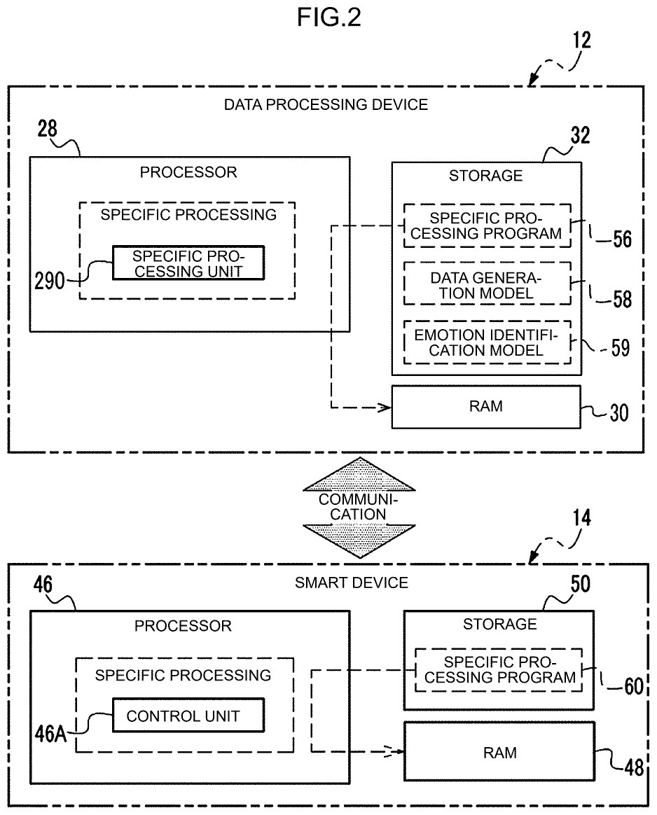

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The online medical care system according to the embodiment of the present invention is a system designed to solve issues such as insufficient information necessary for diagnosis and the difficulty of dissemination to the elderly generation. This online medical care system utilizes generative AI to perform highly accurate predictions by using unique information of each individual as a key, even with a small number of parameters, and examines methods for service penetration suitable for a super-aging society. For example, when a user receives online medical care, the generative AI receives as input a small number of parameters such as the user's basic information and past medical history. For instance, information such as age, gender, medical history, and current symptoms is input. This information is input to the generative AI. Next, the generative AI analyzes the input information and supplements information necessary for diagnosis using unique information of each individual as a key. The generative AI predicts the user's symptoms based on past medical data and similar case data. For example, it refers to data of patients in the same age group and gender to predict diagnosis results for current symptoms. Furthermore, based on the diagnosis result predicted by the generative AI, appropriate treatment methods or advice are provided to the user. For example, proposals such as medication prescriptions or lifestyle improvement points are made. At this time, the generative AI provides personalized advice by considering the user's unique information. In addition, the generative AI also provides a mechanism for disseminating the service to the parent and child generations. For example, when the parent generation receives online medical care, a message is sent to the child generation prompting them to use a similar service. This promotes the use of online medical care by the entire family. Through this mechanism, the accuracy of diagnosis in online medical care is improved and dissemination to the elderly generation is promoted. Moreover, by disseminating the service to the parent and child generations, penetration of online medical care suitable for a super-aging society is achieved. As a result, the online medical care system can improve the accuracy of diagnosis and promote dissemination to the elderly generation. Specifically, this online medical care system comprises multiple modules such as a reception unit, analysis unit, provision unit, and dissemination unit, which operate in cooperation. The system receives input data sent from user terminals (smartphones, tablets, PCs, etc.) via the reception unit, and preprocesses parameters such as age (integer value), gender (categorical value), medical history (text or coded data), and current symptoms (text or selectable options) into a vector format (e.g., 1×N-dimensional numerical array). The reception unit performs preprocessing such as missing value imputation, one-hot encoding of categorical values, and tokenization of text on these data, and transfers them to the analysis unit. The analysis unit uses, for example, a Transformer-based large language model or a multimodal neural network (capable of handling mixed text and numerical input), and takes as input the user information vector and past medical history tensor (e.g., M×N dimensions, where M is the number of history records). Examples of input include: (1) 70-year-old male with a history of hypertension and “recent shortness of breath”; (2) 85-year-old female with a history of diabetes and “loss of appetite”; (3) 65-year-old male with no medical history and “fever”. The analysis unit searches for similar cases in a high-dimensional space from a past medical database (e.g., hundreds of thousands of case data), and extracts user-specific feature quantities in a feature extraction layer (multi-layer perceptron or attention mechanism). Next, in the diagnosis prediction layer, outputs such as diagnosis labels (e.g., “suspected heart failure”, “suspected infection”), probability distributions (e.g., confidence scores for each diagnosis candidate), and recommended treatment methods (e.g., drug names, structured data of lifestyle guidance) are generated. Examples of output include: (1) probability distribution such as “suspected heart failure: 0.82, suspected infection: 0.12”; (2) treatment proposals such as “continuation of antihypertensive medication, guidance on salt restriction”; (3) advice such as “recommend additional tests”. The provision unit receives these outputs and generates and displays personalized treatment methods or advice on the user terminal using a natural language generation module. Furthermore, the dissemination unit automatically generates and sends messages such as “Would your family also like to use online medical care?” to the child generation's terminal based on the history and usage status when the parent generation receives medical care. These processes are technically characterized by the fact that, unlike conventional human physician interviews, diagnoses, and information transmission to families, AI autonomously executes non-conventional algorithms such as similarity calculation in high-dimensional feature space, rule-based branching, probabilistic inference, and natural language generation. For training the AI model, loss functions such as cross-entropy and MSE are used, and weight optimization is performed by gradient descent. Data augmentation such as synthetic generation of case data and noise addition can also be utilized. As a result, improvements in computer technology itself are realized, such as increased diagnostic accuracy, reduced input burden, promotion of service dissemination at the family unit, improved data management efficiency, and optimized communication load. Application fields include telemedicine, home care, chronic disease management, nursing care support, and health promotion services, and it particularly contributes to improving medical access for the elderly generation and family units.

The online medical care system according to the embodiment comprises a reception unit, an analysis unit, a provision unit, and a dissemination unit. The reception unit receives as input a small number of parameters such as the user's basic information and past medical history. The user's basic information includes, for example, age, gender, medical history, and current symptoms, but is not limited to these examples. The reception unit, for example, receives as input a small number of parameters such as the user's basic information and past medical history when the user receives online medical care. The generative AI receives as input a small number of parameters such as the user's basic information and past medical history. The analysis unit uses generative AI to analyze the information received by the reception unit and supplement information necessary for diagnosis using unique information of each individual as a key. The analysis unit, for example, predicts the user's symptoms based on past medical data and similar case data. The generative AI predicts the user's symptoms based on past medical data and similar case data. For example, it refers to data of patients in the same age group and gender to predict diagnosis results for current symptoms. The provision unit provides appropriate treatment methods or advice based on the diagnosis result predicted by the analysis unit. The provision unit, for example, proposes medication prescriptions or lifestyle improvement points. The generative AI provides personalized advice by considering the user's unique information. The dissemination unit sends a message prompting the child generation to use a similar service when the parent generation receives online medical care. The dissemination unit, for example, sends a message prompting the child generation to use a similar service when the parent generation receives online medical care. This promotes the use of online medical care by the entire family. As a result, the online medical care system according to the embodiment can improve the accuracy of diagnosis and promote dissemination to the elderly generation. Furthermore, by disseminating the service to the parent and child generations, penetration of online medical care suitable for a super-aging society is achieved. Specifically, this online medical care system is configured such that the modules of the reception unit, analysis unit, provision unit, and dissemination unit operate in cooperation. The system receives input data sent from user terminals (smartphones, tablets, PCs, etc.) via the reception unit, and preprocesses parameters such as age (integer value), gender (categorical value), medical history (text or coded data), and current symptoms (text or selectable options) into a 1×N-dimensional numerical array. The reception unit performs preprocessing such as missing value imputation, one-hot encoding of categorical values, and tokenization of text on these data, and transfers them to the analysis unit. The analysis unit uses, for example, a Transformer-based large language model or a multimodal neural network capable of handling mixed text and numerical input, and takes as input the user information vector and past medical history tensor (M×N dimensions, where M is the number of history records). Examples of input include: 70-year-old male with a history of hypertension and “recent shortness of breath”; 85-year-old female with a history of diabetes and “loss of appetite”; 65-year-old male with no medical history and “fever”. The analysis unit searches for similar cases in a high-dimensional space from a past medical database (hundreds of thousands of case data), and extracts user-specific feature quantities in a feature extraction layer (multi-layer perceptron or attention mechanism). In the diagnosis prediction layer, outputs such as diagnosis labels (e.g., “suspected heart failure”, “suspected infection”), probability distributions (confidence scores for each diagnosis candidate), and recommended treatment methods (drug names, structured data of lifestyle guidance) are generated. Examples of output include: probability distribution such as “suspected heart failure: 0.82, suspected infection: 0.12”; treatment proposals such as “continuation of antihypertensive medication, guidance on salt restriction”; advice such as “recommend additional tests”. The provision unit receives these outputs and generates and displays personalized treatment methods or advice on the user terminal using a natural language generation module. The dissemination unit automatically generates and sends messages such as “Would your family also like to use online medical care?” to the child generation's terminal based on the history and usage status when the parent generation receives medical care. These processes are technically characterized by the fact that, unlike conventional human physician interviews, diagnoses, and information transmission to families, AI autonomously executes non-conventional algorithms such as similarity calculation in high-dimensional feature space, rule-based branching, probabilistic inference, and natural language generation. For training the AI model, loss functions such as cross-entropy and MSE are used, and weight optimization is performed by gradient descent. Data augmentation such as synthetic generation of case data and noise addition can also be utilized. As a result, improvements in computer technology itself are realized, such as increased diagnostic accuracy, reduced input burden, promotion of service dissemination at the family unit, improved data management efficiency, and optimized communication load. Application fields include telemedicine, home care, chronic disease management, nursing care support, and health promotion services, and it particularly contributes to improving medical access for the elderly generation and family units.

The reception unit can estimate a user's emotion and adjust the timing of inputting medical information based on the estimated emotion of the user. For example, if the user is feeling stressed, the reception unit prompts the input of medical information at a time when the user can relax. If the user is relaxed, the reception unit may prompt the input of detailed medical information. If the user is in a hurry, the reception unit may prioritize concise input of medical information. By adjusting the timing of inputting medical information according to the user's emotion, the user's burden can be reduced. Emotion estimation is realized by using an emotion engine or generative AI with emotion estimation functionality, for example. The generative AI may be a text generation AI (such as an LLM) or a multimodal generative AI, but is not limited to these examples. Some or all of the above-described processing in the reception unit may be performed using AI or without using AI. For example, the reception unit may input the user's emotion data to the generative AI and have the generative AI perform emotion estimation. Specifically, the reception unit inputs voice data obtained from the user terminal (e.g., 30 seconds of spoken audio, 16 kHz sampling, 1×480,000-dimensional waveform vector), facial expression images (e.g., one face image, 224×224×3-dimensional RGB tensor), and text input (e.g., spoken text such as “I have not been feeling well lately”) to the emotion estimation AI. For voice, a speech emotion recognition model combining a convolutional neural network and a recurrent neural network is used; for images, a facial expression recognition model based on ResNet or Vision Transformer is used; for text, a large language model is used. The AI outputs emotion labels (e.g., “stress”, “relaxation”, “tension”, “in a hurry”) and emotion scores (e.g., stress level 0.75, relaxation level 0.20). Examples of output include “stress: 0.80, relaxation: 0.10” and “tension: 0.60, normal: 0.30”. The reception unit performs threshold judgment on these output values and executes branching processing such as delaying reminders for medical information input when stress level is high, prompting detailed input when relaxation level is high, and displaying a simplified input screen when the user is in a hurry. As a result, a technical effect of reducing the user's psychological burden and lowering the input dropout rate is obtained. Unlike conventional simple time-based reminders, the technical feature is that AI analyzes diverse biological and behavioral data in a high-dimensional feature space and optimizes timing using non-conventional rules. For training the AI model, a dataset of voice, image, and text data with emotion annotations is used, and weight optimization is performed using cross-entropy loss. Application fields include telemedicine reception, health management apps, stress care services, and online medical care for seniors.

The reception unit can analyze a user's past medical history and select an optimal input method. For example, the reception unit proposes an optimal input method based on medical information previously input by the user. The reception unit may also preferentially propose frequently used input methods based on the user's past medical history. Additionally, the reception unit may analyze the user's past medical history and provide an automatic input function to reduce input effort. By selecting the optimal input method based on the user's past medical history, input effort can be reduced. Some or all of the above-described processing in the reception unit may be performed using AI or without using AI. For example, the reception unit may input the user's past medical history data to the generative AI and have the generative AI select the optimal input method. Specifically, the reception unit inputs a medical history database saved for each user (e.g., records of the past 12 medical visits, structured data for each visit including age, symptoms, input method, input time required, number of input errors, etc., 12×10-dimensional table) to the AI. For handling historical series, an LSTM or Transformer-based time series analysis model is used. The AI extracts features such as “voice input is frequently used”, “few errors with option input”, and “long time required for text input”, and recommends the optimal input interface (e.g., voice input, option input, text input, image upload, etc.). Examples of output include labels such as “voice input recommended”, “option input recommended”, “auto-completion enabled”, and recommendation scores (e.g., voice input 0.85, option input 0.10). Based on these outputs, the reception unit automatically switches the input screen on the user terminal or enables functions such as auto-completion of past input content. As a result, a technical effect of providing an optimized input experience for each user and reducing input errors and input dropout is obtained. Unlike conventional uniform input screen presentation, the technical feature is that AI analyzes historical data in a high-dimensional feature space and optimizes the interface using non-conventional rules. For training the AI model, labeled data of input history, satisfaction, and error rates for each user is used, and loss functions such as MSE and cross-entropy are utilized. Application fields include telemedicine reception, health management apps for seniors, and support systems for people with disabilities.

The reception unit can perform filtering based on the user's current health condition or lifestyle habits when inputting medical information. For example, the reception unit inputs only relevant medical information based on the user's current health condition. The reception unit may also prioritize necessary medical information by considering the user's lifestyle habits. Additionally, the reception unit may filter out unnecessary medical information based on the user's health condition or lifestyle habits. By filtering medical information based on the user's health condition or lifestyle habits, only necessary information can be input. Some or all of the above-described processing in the reception unit may be performed using AI or without using AI. For example, the reception unit may input the user's health condition or lifestyle habit data to the generative AI and have the generative AI perform filtering. Specifically, the reception unit inputs health condition data obtained from the user terminal (e.g., time-series numerical vectors such as blood pressure, blood glucose, weight, steps, sleep time, 7×5 dimensions) and lifestyle habit data (e.g., categorical values or text such as smoking status, drinking frequency, exercise habits, dietary content) to the AI. A multimodal neural network capable of handling mixed numerical, categorical, and text input is used as the AI model. The AI extracts features such as “hypertension tendency”, “lack of exercise”, “lack of sleep” from the input data and outputs relevance scores for each medical information item (e.g., blood pressure management information 0.90, exercise guidance information 0.80, sleep guidance information 0.70). Examples of output include filtering labels such as “blood pressure management information prioritized”, “exercise guidance information prioritized”, “unnecessary item: smoking history”, and display/non-display flags for each item. Based on these outputs, the reception unit hides unnecessary items on the input screen and highlights only necessary items. As a result, a technical effect of greatly reducing the user's input burden and preventing confusion due to input errors or information overload is obtained. Unlike conventional uniform item presentation, the technical feature is that AI analyzes multidimensional data and optimizes item selection using non-conventional rules. For training the AI model, labeled data of health condition, lifestyle habits, and medical item selection history is used, and weight optimization is performed using cross-entropy loss. Application fields include telemedicine reception, health management apps, and lifestyle disease prevention services.

The reception unit can estimate a user's emotion and determine the priority of medical information to be input based on the estimated emotion of the user. For example, if the user is feeling stressed, the reception unit prioritizes the input of important medical information. If the user is relaxed, the reception unit may prioritize the input of detailed medical information. If the user is in a hurry, the reception unit may prioritize the input of concise medical information. By determining the priority of medical information to be input according to the user's emotion, important information can be input preferentially. Emotion estimation is realized by using an emotion engine or generative AI with emotion estimation functionality, for example. The generative AI may be a text generation AI (such as an LLM) or a multimodal generative AI, but is not limited to these examples. Some or all of the above-described processing in the reception unit may be performed using AI or without using AI. For example, the reception unit may input the user's emotion data to the generative AI and have the generative AI perform emotion estimation. Specifically, the reception unit inputs voice, image, and text data obtained from the user terminal (e.g., spoken audio, face image, input text) to the emotion estimation AI, and outputs emotion labels (e.g., “stress”, “relaxation”, “in a hurry”) and emotion scores (e.g., stress level 0.70). A multimodal neural network capable of handling mixed voice, image, and text input is used as the AI model. The AI outputs priority scores for each medical information item based on the emotion score (e.g., chief complaint input 0.95, medical history input 0.80, lifestyle habit input 0.60). Examples of output include priority labels such as “chief complaint input prioritized”, “medical history input prioritized”, “detailed input omitted”, and display order for each item. Based on these outputs, the reception unit displays important items at the top of the input screen and postpones detailed items, among other branching processes. As a result, a technical effect of reducing the user's psychological burden and preventing omission of important information is obtained. Unlike conventional uniform input order presentation, the technical feature is that AI analyzes diverse emotion data and optimizes priority using non-conventional rules. For training the AI model, labeled data of emotion states and input order history is used, and weight optimization is performed using cross-entropy loss. Application fields include telemedicine reception, stress care services, and health management apps for seniors.

The reception unit can preferentially input highly relevant information based on the user's geographic location information when inputting medical information. For example, the reception unit prioritizes the input of relevant medical information based on the user's current location. The reception unit may also prioritize the input of region-specific medical information by considering the user's geographic location information. Additionally, the reception unit may prioritize the input of necessary medical information based on the user's geographic location information. By considering the user's geographic location information when inputting medical information, region-specific information can be input preferentially. Some or all of the above-described processing in the reception unit may be performed using AI or without using AI. For example, the reception unit may input the user's geographic location information data to the generative AI and have the generative AI select highly relevant information. Specifically, the reception unit inputs geographic information obtained from the user terminal (e.g., GPS coordinates such as latitude 35.6, longitude 139.7 as a 2-dimensional numerical vector, postal code, residential area code) to the AI. As the AI model, a graph neural network that learns the relationship between geographic information and medical items, or an encoder that embeds geographic features, is used. The AI estimates region-specific disease risks (e.g., pollen allergy prevalent areas, infectious disease prevalent areas, areas with high incidence of specific lifestyle diseases) and regional medical resources (e.g., nearby medical institutions, pharmacies, testing facilities) from the input geographic information, and outputs relevance scores for each medical information item (e.g., pollen allergy information 0.90, infectious disease information 0.80). Examples of output include labels such as “pollen allergy information prioritized”, “infectious disease information prioritized”, “regional medical institution information displayed”, and display order for each item. Based on these outputs, the reception unit displays region-specific items at the top of the input screen and hides unnecessary items. As a result, a technical effect of realizing medical information input optimized for the user's residential area and reducing information excess or input burden is obtained. Unlike conventional uniform item presentation, the technical feature is that AI analyzes geographic information in a high-dimensional space and optimizes item selection using non-conventional rules. For training the AI model, labeled data of geographic information and medical item selection history is used, and weight optimization is performed using cross-entropy loss. Application fields include telemedicine reception, regional medical cooperation systems, and health management services during infectious disease outbreaks.

The reception unit can analyze a user's social media activity and input relevant information when inputting medical information. For example, the reception unit inputs relevant medical information based on the user's social media activity. The reception unit may also analyze the user's social media activity and input necessary medical information. Additionally, the reception unit may supplement medical information by considering the user's social media activity. By analyzing the user's social media activity and inputting medical information, highly relevant information can be input. Some or all of the above-described processing in the reception unit may be performed using AI or without using AI. For example, the reception unit may input the user's social media activity data to the generative AI and have the generative AI select relevant information. Specifically, the reception unit inputs social media activity data obtained from the user terminal (e.g., text of up to 100 posts in the past month, up to 500 characters per post, 100×500-dimensional string array), time-series data of post timestamps, and accompanying image data (e.g., one image per post, 224×224×3-dimensional RGB image tensor) to the AI. For text analysis, a Transformer-based large language model is used; for image analysis, a CNN or Vision Transformer is used; for time-series analysis, a multimodal neural network combining LSTM or time-series encoder is used. The AI extracts keywords related to health condition (e.g., “feeling unwell”, “headache”, “lack of exercise”) and lifestyle habits (e.g., “staying up late”, “eating out”, “exercise”) from the input post text, determines dietary content and exercise status from images, and estimates lifestyle rhythm tendencies (e.g., frequent late-night posts, morning type) from post times. The AI integrates these features and outputs relevance scores for each medical information item (e.g., sleep habit information 0.85, dietary content information 0.80, exercise habit information 0.75) and input completion labels (e.g., “tendency for lack of sleep”, “tendency for lack of exercise”). Examples of output include labels such as “sleep habit information prioritized”, “automatic completion of dietary content”, “exercise habit input recommended”, and display/non-display flags for each item. Based on these outputs, the reception unit displays highly relevant items at the top of the medical information input screen, hides unnecessary items, or enables functions to automatically supplement medical information from post content. AI processing is technically characterized by autonomous execution of semantic analysis in high-dimensional feature space, integrated feature extraction from images, text, and time-series, and non-conventional rule-based branching, unlike conventional manual post viewing or keyword search. For training the AI model, a labeled dataset of social media activity and health condition/medical information input history is used, and weight optimization is performed using cross-entropy loss or MSE loss. Data augmentation such as paraphrase generation of post text and noise addition to images can also be utilized. As a result, improvements in computer technology itself are realized, such as increased accuracy of medical information input, reduced user burden, early grasp of health condition, improved data management efficiency, and optimized communication load. Application fields include telemedicine reception, health management apps, lifestyle disease prevention services, and health promotion services for young people, and it particularly contributes to improving medical access for social media user groups.

The analysis unit can estimate a user's emotion and adjust the method of presenting analysis based on the estimated emotion of the user. For example, if the user is nervous, the analysis unit provides a simple and highly visible analysis result. If the user is relaxed, the analysis unit may provide a detailed analysis result. If the user is in a hurry, the analysis unit may provide an analysis result that focuses on the main points. By adjusting the method of presenting analysis according to the user's emotion, analysis results that are easy for the user to understand can be provided. Emotion estimation is realized by using an emotion engine or generative AI with emotion estimation functionality, for example. The generative AI may be a text generation AI (such as an LLM) or a multimodal generative AI, but is not limited to these examples. Some or all of the above-described processing in the analysis unit may be performed using AI or without using AI. For example, the analysis unit may input the user's emotion data to the generative AI and have the generative AI perform emotion estimation. Specifically, the analysis unit inputs voice data obtained from the user terminal (e.g., 30 seconds of spoken audio, 16 kHz sampling, 1×480,000-dimensional waveform vector), facial expression images (e.g., one face image, 224×224×3-dimensional RGB tensor), and text input (e.g., spoken text such as “I am anxious about the result”) to the emotion estimation AI. For voice, a speech emotion recognition model combining CNN and RNN is used; for images, a facial expression recognition model based on ResNet or Vision Transformer is used; for text, a large language model is used. The AI outputs emotion labels (e.g., “nervous”, “relaxed”, “in a hurry”) and emotion scores (e.g., nervousness level 0.80, relaxation level 0.10). Examples of output include “nervous: 0.75, relaxed: 0.15” and “in a hurry: 0.60, normal: 0.30”. The analysis unit performs threshold judgment on these output values and executes branching processing such as displaying analysis results in a simple format such as bullet points or graphs when nervousness level is high, displaying analysis results with detailed numbers and explanations when relaxation level is high, and displaying only the main points in short sentences when the user is in a hurry. AI processing is technically characterized by optimizing the method of presenting analysis using non-conventional rules by analyzing the user's emotional state from diverse biological and behavioral data in a high-dimensional feature space, unlike conventional uniform analysis result presentation. For training the AI model, a dataset of voice, image, and text data with emotion annotations and labeled data of analysis result presentation history is used, and weight optimization is performed using cross-entropy loss. As a result, improvements in computer technology itself are realized, such as increased understanding of analysis results, improved user satisfaction, reduced input dropout rate, and improved data management efficiency. Application fields include telemedicine analysis result presentation, health management apps, stress care services, and online medical care for seniors.

The analysis unit can adjust the level of detail of analysis based on the importance of medical information during analysis. For example, the analysis unit performs detailed analysis for important medical information. The analysis unit may also perform concise analysis for less important medical information. Additionally, the analysis unit may adjust the level of detail of analysis according to the importance of medical information. By adjusting the level of detail of analysis according to the importance of medical information, detailed analysis can be performed for important information. Some or all of the above-described processing in the analysis unit may be performed using AI or without using AI. For example, the analysis unit may input importance score data of medical information to the generative AI and have the generative AI adjust the level of detail. Specifically, the analysis unit inputs importance scores assigned to each medical information item (e.g., chief complaint 1.0, medical history 0.8, lifestyle habits 0.6 as continuous values from 0 to 1, 1×N-dimensional vector) to the AI. As the AI model, a rule-based branching network or a conditional natural language generation model that controls analysis templates and output content based on importance scores is used. The AI generates detailed analysis (e.g., comparison with past data, detailed analysis of risk factors, explanation of recommended treatment methods) for items with high importance, and concise analysis summarizing only the main points for items with low importance. Examples of output include labels such as “chief complaint: detailed analysis+explanation”, “medical history: simple analysis”, “lifestyle habits: main points only”, and parameters controlling the length and content level of analysis for each item. Based on these outputs, the analysis unit displays detailed analysis results for important items and concise results for other items on the user terminal. AI processing is technically characterized by optimizing the level of detail using non-conventional rules by analyzing the importance of medical information in a high-dimensional feature space, unlike conventional uniform analysis detail settings. For training the AI model, labeled data of medical information importance and analysis result detail level is used, and weight optimization is performed using cross-entropy loss or MSE loss. As a result, improvements in computer technology itself are realized, such as increased analysis accuracy, improved user satisfaction, prevention of information overload, and improved data management efficiency. Application fields include telemedicine analysis, health management apps, and chronic disease management services.

The analysis unit can apply different analysis algorithms according to the category of medical information during analysis. For example, the analysis unit selects the optimal analysis algorithm according to the category of medical information. The analysis unit may also apply different analysis algorithms for each category of medical information. Additionally, the analysis unit may adjust the analysis algorithm based on the category of medical information. By applying the optimal analysis algorithm according to the category of medical information, the accuracy of analysis can be improved. Some or all of the above-described processing in the analysis unit may be performed using AI or without using AI. For example, the analysis unit may input category data of medical information to the generative AI and have the generative AI apply the analysis algorithm. Specifically, the analysis unit inputs category labels assigned to each medical information item (e.g., chief complaint, medical history, lifestyle habits, test values as categorical values, 1×N-dimensional category array) to the AI. As the AI model, a multitask neural network that automatically selects and applies different analysis algorithms for each category (e.g., natural language processing model for chief complaint, regression analysis model for test values, CNN-based image analysis model for image data, time series analysis model for lifestyle habits) or a category-conditioned algorithm selection network is used. The AI calls the optimal analysis algorithm according to the input category and generates optimized analysis results for each category (e.g., chief complaint→text summary, test values→anomaly detection, image→lesion area extraction, lifestyle habits→risk score calculation). Examples of output include labels such as “chief complaint: summary+risk estimation”, “test values: anomaly detection”, “image: lesion extraction”, “lifestyle habits: risk score”, and analysis results for each category. Based on these outputs, the analysis unit displays optimized analysis results for each category on the user terminal. AI processing is technically characterized by optimizing algorithm selection using non-conventional rules by analyzing the category of medical information in a high-dimensional feature space, unlike conventional uniform algorithm application. For training the AI model, labeled data of input data for each category and optimal algorithm/analysis result is used, and weight optimization is performed using cross-entropy loss or MSE loss. As a result, improvements in computer technology itself are realized, such as increased analysis accuracy, improved computational efficiency, prevention of information overload, and improved data management efficiency. Application fields include telemedicine analysis, health management apps, and test value analysis services.

The analysis unit can adjust the length of analysis based on the estimated emotion of the user. For example, if the user is nervous, the analysis unit provides a short analysis result that focuses on the main points. If the user is relaxed, the analysis unit may provide a detailed analysis result. If the user is in a hurry, the analysis unit may provide a concise analysis result. By adjusting the length of analysis according to the user's emotion, analysis results of appropriate length can be provided to the user. Emotion estimation is realized by using an emotion engine or generative AI with emotion estimation functionality, for example. The generative AI may be a text generation AI (such as an LLM) or a multimodal generative AI, but is not limited to these examples. Some or all of the above-described processing in the analysis unit may be performed using AI or without using AI. For example, the analysis unit may input the user's emotion data to the generative AI and have the generative AI perform emotion estimation. Specifically, the analysis unit inputs voice data obtained from the user terminal (e.g., 30 seconds of spoken audio, 16 kHz sampling, 1×480,000-dimensional waveform vector), facial expression images (e.g., one face image, 224×224×3-dimensional RGB tensor), and text input (e.g., spoken text such as “I am anxious about the result”) to the emotion estimation AI. For voice, a speech emotion recognition model combining CNN and RNN is used; for images, a facial expression recognition model based on ResNet or Vision Transformer is used; for text, a large language model is used. The AI outputs emotion labels (e.g., “nervous”, “relaxed”, “in a hurry”) and emotion scores (e.g., nervousness level 0.80, relaxation level 0.10). Examples of output include “nervous: 0.75, relaxed: 0.15” and “in a hurry: 0.60, normal: 0.30”. The analysis unit performs threshold judgment on these output values and executes branching processing such as displaying only the main points in short sentences when nervousness level is high, displaying detailed numbers and explanations in long sentences when relaxation level is high, and displaying only concise summaries when the user is in a hurry. AI processing is technically characterized by optimizing the length using non-conventional rules by analyzing the user's emotional state from diverse biological and behavioral data in a high-dimensional feature space, unlike conventional uniform analysis result length settings. For training the AI model, a dataset of voice, image, and text data with emotion annotations and labeled data of analysis result presentation history is used, and weight optimization is performed using cross-entropy loss. As a result, improvements in computer technology itself are realized, such as increased understanding of analysis results, improved user satisfaction, reduced input dropout rate, and improved data management efficiency. Application fields include telemedicine analysis result presentation, health management apps, stress care services, and online medical care for seniors.

The analysis unit can determine the priority of analysis based on the timing of submission of medical information during analysis. For example, the analysis unit determines the priority of analysis based on the timing of submission of medical information. The analysis unit may also preferentially analyze newly submitted medical information. Additionally, the analysis unit may postpone the analysis of older medical information. By determining the priority of analysis based on the timing of submission of medical information, the latest information can be analyzed preferentially. Some or all of the above-described processing in the analysis unit may be performed using AI or without using AI. For example, the analysis unit may input submission timing data of medical information to the generative AI and have the generative AI determine the priority. Specifically, the analysis unit inputs submission time data assigned to each medical information item (e.g., UNIX timestamp, 1×N-dimensional numerical array) to the AI. As the AI model, a rule-based priority determination network or a time series analysis model that combines submission time and urgency/importance of medical information is used. The AI assigns a high priority score (e.g., 0.95) to newly submitted information and a low priority score (e.g., 0.30) to older information, and outputs analysis order labels (e.g., “newly submitted prioritized”, “older information postponed”) and analysis queue order. Examples of output include labels such as “chief complaint (new): priority 0.95”, “medical history (one week ago): priority 0.40”, and analysis order lists. Based on these outputs, the analysis unit executes analysis in order from the latest medical information and postpones older information. AI processing is technically characterized by optimizing priority using non-conventional rules by analyzing submission time and urgency in a high-dimensional feature space, unlike conventional uniform FIFO methods. For training the AI model, labeled data of submission time, urgency, and analysis order history is used, and weight optimization is performed using cross-entropy loss or MSE loss. As a result, improvements in computer technology itself are realized, such as increased immediacy of analysis, faster emergency response, and improved data management efficiency. Application fields include telemedicine analysis, emergency medical reception, and health management apps.

The analysis unit can adjust the order of analysis based on the relevance of medical information during analysis. For example, the analysis unit adjusts the order of analysis based on the relevance of medical information. The analysis unit may also preferentially analyze highly relevant medical information. Additionally, the analysis unit may postpone the analysis of less relevant medical information. By adjusting the order of analysis based on the relevance of medical information, highly relevant information can be analyzed preferentially. Some or all of the above-described processing in the analysis unit may be performed using AI or without using AI. For example, the analysis unit may input relevance data of medical information to the generative AI and have the generative AI adjust the order. Specifically, the analysis unit inputs relevance scores calculated for each medical information item (e.g., relevance between chief complaint and medical history 0.90, relevance between chief complaint and lifestyle habits 0.60 as continuous values from 0 to 1, N×N-dimensional relevance matrix) to the AI. As the AI model, a graph neural network that optimizes analysis order based on the relevance matrix or an order determination algorithm based on relevance is used. The AI groups highly relevant information and outputs order lists for preferential analysis (e.g., “chief complaint→medical history→lifestyle habits”) and analysis order labels (e.g., “high relevance group prioritized”, “low relevance group postponed”). Examples of output include labels such as “chief complaint and medical history group prioritized”, “lifestyle habits postponed”, and analysis order lists. Based on these outputs, the analysis unit executes analysis in order from highly relevant information and postpones less relevant information. AI processing is technically characterized by optimizing order using non-conventional rules by analyzing relevance between medical information in a high-dimensional feature space, unlike conventional uniform order settings. For training the AI model, labeled data of relevance between medical information and analysis order history is used, and weight optimization is performed using cross-entropy loss or MSE loss. As a result, improvements in computer technology itself are realized, such as increased analysis efficiency, ensured comprehensiveness of information, and improved data management efficiency. Application fields include telemedicine analysis, health management apps, and chronic disease management services.

The provision unit can estimate a user's emotion and adjust the method of presenting a treatment method or advice to be provided based on the estimated emotion of the user. For example, if the user is nervous, the provision unit provides a simple and highly visible method of presentation. If the user is relaxed, the provision unit may provide a method of presentation that includes detailed information. If the user is in a hurry, the provision unit may provide a method of presentation that focuses on the main points. By adjusting the method of presenting a treatment method or advice according to the user's emotion, information that is easy for the user to understand can be provided. Emotion estimation is realized by using an emotion engine or generative AI with emotion estimation functionality, for example. The generative AI may be a text generation AI (such as an LLM) or a multimodal generative AI, but is not limited to these examples. Some or all of the above-described processing in the provision unit may be performed using AI or without using AI. For example, the provision unit may input the user's emotion data to the generative AI and have the generative AI perform emotion estimation. Specifically, the provision unit inputs voice data obtained from the user terminal (e.g., 30 seconds of spoken audio, 16 kHz sampling, 1×480,000-dimensional waveform vector), facial expression images (e.g., one face image, 224×224×3-dimensional RGB tensor), and text input (e.g., spoken text such as “I am anxious about the diagnosis result”) to the emotion estimation AI. For voice data, a speech emotion recognition model combining a convolutional neural network and a recurrent neural network is applied; for image data, a facial expression recognition model based on ResNet or Vision Transformer is applied; for text data, a large language model is applied. The provision unit receives emotion labels (e.g., “nervous”, “relaxed”, “in a hurry”) and emotion scores (e.g., nervousness level 0.80, relaxation level 0.10) as output from the AI. Examples of output include “nervous: 0.75, relaxed: 0.15” and “in a hurry: 0.60, normal: 0.30”. The provision unit performs threshold judgment on these output values and executes branching processing such as displaying treatment methods or advice in a simple format such as short sentences, bullet points, or pictograms when nervousness level is high, generating long advice including detailed numbers and explanations when relaxation level is high, and displaying only the main points in short sentences when the user is in a hurry. The provision unit inputs emotion labels and scores as control parameters to the natural language generation module and dynamically controls the style, length, level of detail, and method of presentation of the generated text. For example, when nervousness level is high, a short sentence such as “Today's diagnosis result is normal. Please rest assured.” is generated; when relaxation level is high, a detailed explanation such as “Today's diagnosis result is within the normal range. Your blood pressure is more stable than last time, and lifestyle improvements are having an effect. Please continue regular exercise and a balanced diet.” is generated; when the user is in a hurry, a short sentence such as “Diagnosis result: no abnormality. Next checkup is recommended in one month.” is generated. These processes are technically characterized by optimizing the method of presentation using non-conventional rules by analyzing diverse biological and behavioral data in a high-dimensional feature space, unlike conventional uniform advice presentation. For training the AI model, a dataset of voice, image, and text data with emotion annotations and labeled data of advice presentation history is used, and weight optimization is performed using cross-entropy loss. As a result, improvements in computer technology itself are realized, such as increased understanding of information, improved user satisfaction, reduced input dropout rate, and improved data management efficiency. Application fields include telemedicine advice presentation, health management apps, stress care services, and online medical care for seniors.

The provision unit can adjust the level of detail of provision based on the importance of the diagnosis result during provision. For example, the provision unit provides detailed information for important diagnosis results. The provision unit may also provide concise information for less important diagnosis results. Additionally, the provision unit may adjust the level of detail of provision according to the importance of the diagnosis result. By adjusting the level of detail of provision according to the importance of the diagnosis result, detailed provision can be made for important information. Some or all of the above-described processing in the provision unit may be performed using AI or without using AI. For example, the provision unit may input importance score data of diagnosis results to the generative AI and have the generative AI adjust the level of detail. Specifically, the provision unit inputs importance scores assigned to each diagnosis result (e.g., chief complaint 1.0, medical history 0.8, lifestyle habits 0.6 as continuous values from 0 to 1, 1×N-dimensional vector) to the AI. The provision unit uses a rule-based branching network or a conditional natural language generation model that controls provision templates and output content based on importance scores. The AI generates detailed provision (e.g., comparison with past data, detailed analysis of risk factors, explanation of recommended treatment methods) for items with high importance, and concise information summarizing only the main points for items with low importance. Examples of output include labels such as “chief complaint: detailed provision+explanation”, “medical history: simple provision”, “lifestyle habits: main points only”, and parameters controlling the length and content level of provision for each item. Based on these outputs, the provision unit displays detailed information for important items and concise information for other items on the user terminal. AI processing is technically characterized by optimizing the level of detail using non-conventional rules by analyzing the importance of diagnosis results in a high-dimensional feature space, unlike conventional uniform provision detail settings. For training the AI model, labeled data of diagnosis result importance and provision detail level is used, and weight optimization is performed using cross-entropy loss or MSE loss. As a result, improvements in computer technology itself are realized, such as increased provision accuracy, improved user satisfaction, prevention of information overload, and improved data management efficiency. Application fields include telemedicine advice provision, health management apps, and chronic disease management services.

The provision unit can apply different provision algorithms according to the category of the diagnosis result during provision. For example, the provision unit selects the optimal provision algorithm according to the category of the diagnosis result. The provision unit may also apply different provision algorithms for each category of the diagnosis result. Additionally, the provision unit may adjust the provision algorithm based on the category of the diagnosis result. By applying the optimal provision algorithm according to the category of the diagnosis result, the accuracy of provision can be improved. Some or all of the above-described processing in the provision unit may be performed using AI or without using AI. For example, the provision unit may input category data of diagnosis results to the generative AI and have the generative AI apply the provision algorithm. Specifically, the provision unit inputs category labels assigned to each diagnosis result (e.g., chief complaint, medical history, lifestyle habits, test values as categorical values, 1×N-dimensional category array) to the AI. The provision unit uses a multitask neural network that automatically selects and applies different provision algorithms for each category (e.g., natural language generation model for chief complaint, graph generation model for test values, image caption generation model for image data, behavior proposal generation model for lifestyle habits) or a category-conditioned algorithm selection network. The AI calls the optimal provision algorithm according to the input category and generates optimized provision results for each category (e.g., chief complaint→text summary, test values→explanation with graph, image→caption generation, lifestyle habits→behavior proposal). Examples of output include labels such as “chief complaint: summary+risk estimation”, “test values: graph generation”, “image: caption generation”, “lifestyle habits: behavior proposal”, and provision results for each category. Based on these outputs, the provision unit displays optimized provision results for each category on the user terminal. AI processing is technically characterized by optimizing algorithm selection using non-conventional rules by analyzing the category of diagnosis results in a high-dimensional feature space, unlike conventional uniform algorithm application. For training the AI model, labeled data of input data for each category and optimal algorithm/provision result is used, and weight optimization is performed using cross-entropy loss or MSE loss. As a result, improvements in computer technology itself are realized, such as increased provision accuracy, improved computational efficiency, prevention of information overload, and improved data management efficiency. Application fields include telemedicine advice provision, health management apps, and test value analysis services.

The provision unit can adjust the length of a treatment method or advice to be provided based on the estimated emotion of the user. For example, if the user is nervous, the provision unit provides a short treatment method or advice that focuses on the main points. If the user is relaxed, the provision unit may provide a detailed treatment method or advice. If the user is in a hurry, the provision unit may provide a concise treatment method or advice. By adjusting the length of a treatment method or advice according to the user's emotion, information of appropriate length can be provided to the user. Emotion estimation is realized by using an emotion engine or generative AI with emotion estimation functionality, for example. The generative AI may be a text generation AI (such as an LLM) or a multimodal generative AI, but is not limited to these examples. Some or all of the above-described processing in the provision unit may be performed using AI or without using AI. For example, the provision unit may input the user's emotion data to the generative AI and have the generative AI perform emotion estimation. Specifically, the provision unit inputs voice data obtained from the user terminal (e.g., 30 seconds of spoken audio, 16 kHz sampling, 1×480,000-dimensional waveform vector), facial expression images (e.g., one face image, 224×224×3-dimensional RGB tensor), and text input (e.g., spoken text such as “I am anxious about the diagnosis result”) to the emotion estimation AI. For voice data, a speech emotion recognition model combining a convolutional neural network and a recurrent neural network is applied; for image data, a facial expression recognition model based on ResNet or Vision Transformer is applied; for text data, a large language model is applied. The AI outputs emotion labels (e.g., “nervous”, “relaxed”, “in a hurry”) and emotion scores (e.g., nervousness level 0.80, relaxation level 0.10). Examples of output include “nervous: 0.75, relaxed: 0.15” and “in a hurry: 0.60, normal: 0.30”. The provision unit performs threshold judgment on these output values and executes branching processing such as displaying only the main points in short sentences when nervousness level is high, generating long advice including detailed numbers and explanations when relaxation level is high, and displaying only concise summaries when the user is in a hurry. AI processing is technically characterized by optimizing the length using non-conventional rules by analyzing the user's emotional state from diverse biological and behavioral data in a high-dimensional feature space, unlike conventional uniform advice length settings. For training the AI model, a dataset of voice, image, and text data with emotion annotations and labeled data of advice presentation history is used, and weight optimization is performed using cross-entropy loss. As a result, improvements in computer technology itself are realized, such as increased understanding of advice, improved user satisfaction, reduced input dropout rate, and improved data management efficiency. Application fields include telemedicine advice presentation, health management apps, stress care services, and online medical care for seniors.

The provision unit can determine the priority of provision based on the timing of submission of the diagnosis result during provision. For example, the provision unit determines the priority of provision based on the timing of submission of the diagnosis result. The provision unit may also preferentially provide newly submitted diagnosis results. Additionally, the provision unit may postpone the provision of older diagnosis results. By determining the priority of provision based on the timing of submission of the diagnosis result, the latest information can be provided preferentially. Some or all of the above-described processing in the provision unit may be performed using AI or without using AI. For example, the provision unit may input submission timing data of diagnosis results to the generative AI and have the generative AI determine the priority. Specifically, the provision unit inputs submission time data assigned to each diagnosis result (e.g., UNIX timestamp, 1×N-dimensional numerical array) to the AI. The provision unit uses a rule-based priority determination network or a time series analysis model that combines submission time and urgency/importance of diagnosis results. The AI assigns a high priority score (e.g., 0.95) to newly submitted information and a low priority score (e.g., 0.30) to older information, and outputs provision order labels (e.g., “newly submitted prioritized”, “older information postponed”) and provision queue order. Examples of output include labels such as “chief complaint (new): priority 0.95”, “medical history (one week ago): priority 0.40”, and provision order lists. Based on these outputs, the provision unit executes provision in order from the latest diagnosis result and postpones older information. AI processing is technically characterized by optimizing priority using non-conventional rules by analyzing submission time and urgency in a high-dimensional feature space, unlike conventional uniform FIFO methods. For training the AI model, labeled data of submission time, urgency, and provision order history is used, and weight optimization is performed using cross-entropy loss or MSE loss. As a result, improvements in computer technology itself are realized, such as increased immediacy of provision, faster emergency response, and improved data management efficiency. Application fields include telemedicine advice provision, emergency medical reception, and health management apps.

The provision unit can adjust the order of provision based on the relevance of diagnosis results at the time of provision. For example, the provision unit adjusts the order of provision based on the relevance of diagnosis results. Additionally, the provision unit can preferentially provide highly relevant diagnosis results, or postpone the provision of less relevant diagnosis results. By adjusting the order of provision based on the relevance of diagnosis results, highly relevant information can be provided preferentially. Some or all of the above-described processing in the provision unit may be performed using AI, or may be performed without using AI. For example, the provision unit can input relevance data of diagnosis results into a generative AI and have the generative AI execute the adjustment of the order. Specifically, the provision unit inputs relevance scores calculated for each diagnosis result (e.g., a continuous value vector from 0 to 1 such as a relevance of 0.90 between chief complaint and past medical history, a relevance of 0.60 between chief complaint and lifestyle habits, or an N×N dimensional relevance matrix) into the AI. The provision unit uses a graph neural network that optimizes the order of provision based on the relevance matrix, or an order determination algorithm based on relevance. The AI groups highly relevant information and outputs an order list for preferential provision (e.g., “chief complaint→past medical history→lifestyle habits”) or order labels (e.g., “high relevance group prioritized”, “low relevance group postponed”). Examples of output include labels such as “chief complaint and past medical history group prioritized”, “lifestyle habits postponed”, or provision order lists. Based on these outputs, the provision unit executes provision in order from highly relevant information, postponing less relevant information. Unlike conventional uniform order settings, AI processing is characterized by analyzing the relevance between diagnosis results in a high-dimensional feature space and optimizing the order using non-conventional rules. For training the AI model, labeled data of relevance between diagnosis results and provision order history are used, and weights are optimized using cross-entropy loss or MSE loss. This enables improvements in provision efficiency, ensuring comprehensiveness of information, efficient data management, and overall improvement of computer technology. Application fields include remote medical advice provision, health management apps, and chronic disease management services.

The dissemination unit can estimate a user's emotion and adjust the method of presenting dissemination messages based on the estimated emotion. For example, when the user is nervous, the dissemination unit provides simple and highly visible dissemination messages. When the user is relaxed, the dissemination unit can provide dissemination messages containing detailed information. When the user is in a hurry, the dissemination unit can provide dissemination messages that focus on key points. By adjusting the method of presenting dissemination messages according to the user's emotion, messages that are easy for the user to understand can be provided. Emotion estimation is realized using, for example, an emotion engine or a generative AI with emotion estimation functionality. The generative AI may be a text generation AI (such as an LLM) or a multimodal generative AI, but is not limited to these examples. Some or all of the above-described processing in the dissemination unit may be performed using AI, or may be performed without using AI. For example, the dissemination unit can input user emotion data into a generative AI and have the generative AI execute emotion estimation. Specifically, the dissemination unit inputs voice data obtained from the user terminal (e.g., 30 seconds of speech audio, 16 kHz sampling, 1×480,000 dimensional waveform vector), facial expression images (e.g., one face image, 224×224×3 dimensional RGB tensor), and text input (e.g., speech text such as “I want to recommend medical care to my family”) into an emotion estimation AI. For voice data, a speech emotion recognition model combining convolutional neural networks and recurrent neural networks is applied; for image data, a facial expression recognition model based on ResNet or Vision Transformer is applied; and for text data, a large language model is applied. The dissemination unit receives emotion labels (e.g., “nervous”, “relaxed”, “in a hurry”) and emotion scores (e.g., nervousness 0.80, relaxation 0.10) as output from the AI. Examples of output include “nervous: 0.75, relaxed: 0.15”, “in a hurry: 0.60, normal: 0.30”, etc. The dissemination unit performs threshold judgment on these output values: if nervousness is high, dissemination messages are displayed in simple formats such as short sentences, bullet points, or pictograms; if relaxation is high, long dissemination messages including detailed figures and explanations are generated; if in a hurry, only key points are summarized in short sentences. The dissemination unit inputs emotion labels and scores as control parameters to a natural language generation module, dynamically controlling the style, length, level of detail, and method of expression of the generated text. For example, if nervousness is high, a short sentence that provides reassurance such as “Your family can also use online medical care. Please rest assured.” is generated; if relaxation is high, a detailed explanation such as “For your family's health management, we will guide you through the details of the online medical care service. We recommend use by all family members.” is generated; if in a hurry, a short sentence focusing only on key points such as “Your family can also use medical care. Details will be provided later.” is generated. Unlike conventional uniform message display, this processing is characterized by the dissemination unit analyzing diverse biometric and behavioral data in a high-dimensional feature space and optimizing the method of expression using non-conventional rules. For training the AI model, emotion-annotated voice, image, and text datasets and labeled data of dissemination message expression history are used, and weights are optimized using cross-entropy loss. This enables improvements in user comprehension, increased service adoption among families, reduced input dropout rates, efficient data management, and overall improvement of computer technology. Application fields include family dissemination of remote medical services, introduction functions of health management apps, and promotion of online medical care for seniors.

The dissemination unit can analyze the medical history of the parent generation at the time of dissemination and select the optimal dissemination method. For example, the dissemination unit selects the optimal dissemination method based on the medical history of the parent generation. The dissemination unit can also propose effective dissemination methods based on the medical history of the parent generation, and customize the content of dissemination messages by analyzing the medical history of the parent generation. By selecting the optimal dissemination method based on the medical history of the parent generation, effective dissemination becomes possible. Some or all of the above-described processing in the dissemination unit may be performed using AI, or may be performed without using AI. For example, the dissemination unit can input medical history data of the parent generation into a generative AI and have the generative AI execute selection of the optimal dissemination method. Specifically, the dissemination unit inputs a medical history database saved for each parent generation (e.g., records of the past 24 medical visits, including structured data for each visit such as diagnosis details, treatment progress, usage frequency, and sharing history with family, 24×15 dimensional table) into the AI. For handling history series, LSTM or Transformer-based time series analysis models are used. The AI extracts features such as “high frequency of medical visits”, “many family sharing histories”, “increased family use for specific diagnoses” from the input data, and recommends optimal dissemination interfaces (e.g., email notifications, in-app banners, push notifications, family group chats) and dissemination timing (e.g., immediately after medical care, weekends, family events). Examples of output include labels such as “push notification recommended”, “family group chat recommended”, “dissemination immediately after medical care recommended”, and recommendation scores (e.g., push notification 0.85, email 0.10). Based on these outputs, the dissemination unit automatically switches dissemination methods and customizes the content of dissemination messages according to the medical details. For example, for parent generations managing chronic diseases, content such as “We recommend regular health checks for your family as well” is generated; for acute diseases, content such as “Please pay attention to changes in your family's health condition” is generated. Unlike conventional uniform dissemination method suggestions, AI processing is characterized by analyzing medical history data in a high-dimensional feature space and optimizing dissemination methods and content using non-conventional rules. For training the AI model, labeled data of medical history and dissemination effectiveness or family usage rates are used, and MSE or cross-entropy is used as the loss function. This enables improvements in dissemination accuracy, promotion of service adoption at the family level, efficient data management, optimization of communication load, and overall improvement of computer technology. Application fields include family dissemination of remote medical services, introduction functions of health management apps, and promotion of family collaboration in chronic disease management.

The dissemination unit can customize dissemination messages based on the current health condition of the parent generation at the time of dissemination. For example, the dissemination unit customizes the content of dissemination messages based on the current health condition of the parent generation. The dissemination unit can also provide appropriate dissemination messages according to the health condition of the parent generation, and adjust the method of presenting dissemination messages in consideration of the health condition of the parent generation. By customizing dissemination messages based on the health condition of the parent generation, appropriate messages can be provided. Some or all of the above-described processing in the dissemination unit may be performed using AI, or may be performed without using AI. For example, the dissemination unit can input health condition data of the parent generation into a generative AI and have the generative AI execute message customization. Specifically, the dissemination unit inputs health condition data of the parent generation (e.g., time series numerical vectors for blood pressure, blood glucose, weight, steps, sleep time, 14×5 dimensions), lifestyle habit data (e.g., smoking status, drinking frequency, exercise habits, dietary content as categorical values or text) into the AI. A multimodal neural network capable of handling mixed inputs of numerical, categorical, and text data is used. The AI extracts features such as “hypertension tendency”, “lack of exercise”, “lack of sleep” from the input data, and outputs relevance scores for each dissemination message item (e.g., blood pressure management information 0.90, exercise guidance information 0.80, sleep guidance information 0.70), as well as customization labels (e.g., “blood pressure management recommended”, “exercise habit improvement recommended”). Examples of output include filtering labels such as “blood pressure management information prioritized”, “exercise guidance information prioritized”, “unnecessary item: smoking history”, and display/non-display flags for each item. Based on these outputs, the dissemination unit hides unnecessary items in dissemination messages and highlights only necessary items. For example, if the parent generation has a tendency toward hypertension, content such as “Please pay attention to blood pressure management for your family as well” is generated; if there is a lack of exercise, content such as “Why not start exercising together as a family?” is generated. Unlike conventional uniform message suggestions, AI processing is characterized by analyzing health condition data in a high-dimensional feature space and optimizing item selection and method of expression using non-conventional rules. For training the AI model, labeled data of health condition, lifestyle habits, and dissemination message content selection history are used, and weights are optimized using cross-entropy loss. This enables improvements in dissemination accuracy, increased health awareness at the family level, efficient data management, optimization of communication load, and overall improvement of computer technology. Application fields include family dissemination of remote medical services, introduction functions of health management apps, and promotion of family collaboration in lifestyle disease prevention.

The dissemination unit can estimate a user's emotion and determine the priority of dissemination messages based on the estimated emotion. For example, when the user is nervous, the dissemination unit preferentially provides important dissemination messages. When the user is relaxed, the dissemination unit can preferentially provide detailed dissemination messages. When the user is in a hurry, the dissemination unit can preferentially provide concise dissemination messages. By determining the priority of dissemination messages according to the user's emotion, important messages can be provided preferentially. Emotion estimation is realized using, for example, an emotion engine or a generative AI with emotion estimation functionality. The generative AI may be a text generation AI (such as an LLM) or a multimodal generative AI, but is not limited to these examples. Some or all of the above-described processing in the dissemination unit may be performed using AI, or may be performed without using AI. For example, the dissemination unit can input user emotion data into a generative AI and have the generative AI execute emotion estimation. Specifically, the dissemination unit inputs voice data obtained from the user terminal (e.g., 30 seconds of speech audio, 16 kHz sampling, 1×480,000 dimensional waveform vector), facial expression images (e.g., one face image, 224×224×3 dimensional RGB tensor), and text input (e.g., speech text such as “I want to recommend medical care to my family”) into an emotion estimation AI. For voice data, a speech emotion recognition model combining convolutional neural networks and recurrent neural networks is applied; for image data, a facial expression recognition model based on ResNet or Vision Transformer is applied; and for text data, a large language model is applied. The dissemination unit receives emotion labels (e.g., “nervous”, “relaxed”, “in a hurry”) and emotion scores (e.g., nervousness 0.80, relaxation 0.10) as output from the AI. Examples of output include “nervous: 0.75, relaxed: 0.15”, “in a hurry: 0.60, normal: 0.30”, etc. The dissemination unit performs threshold judgment on these output values: if nervousness is high, important messages such as “Your family can also receive medical care” are displayed at the top; if relaxation is high, detailed explanations and additional information are preferentially displayed; if in a hurry, concise messages focusing only on key points are preferentially displayed. Unlike conventional uniform priority settings, AI processing is characterized by analyzing emotion data in a high-dimensional feature space and optimizing priority using non-conventional rules. For training the AI model, labeled data of emotional states and dissemination message priority history are used, and weights are optimized using cross-entropy loss. This enables improvements in the efficiency of important information transmission, increased user satisfaction, reduced input dropout rates, efficient data management, and overall improvement of computer technology. Application fields include family dissemination of remote medical services, introduction functions of health management apps, and promotion of online medical care for seniors.

The dissemination unit can select the optimal dissemination method by considering the geographic location information of the parent generation at the time of dissemination. For example, the dissemination unit selects the optimal dissemination method based on the geographic location information of the parent generation. The dissemination unit can also propose effective dissemination methods by considering the geographic location information of the parent generation, and customize the content of dissemination messages based on the geographic location information of the parent generation. By considering the geographic location information of the parent generation and selecting the optimal dissemination method, effective dissemination becomes possible. Some or all of the above-described processing in the dissemination unit may be performed using AI, or may be performed without using AI. For example, the dissemination unit can input geographic location information data of the parent generation into a generative AI and have the generative AI execute selection of the optimal dissemination method. Specifically, the dissemination unit inputs the GPS coordinates of the parent generation (e.g., latitude 35.6, longitude 139.7 as a 2-dimensional numerical vector), postal code, residential area code, and other geographic information into the AI. A graph neural network that learns the relationship between geographic information and dissemination methods, or an encoder that embeds geographic features, is used. The AI estimates region-specific medical resources (e.g., nearby medical institutions, pharmacies, testing facilities), regional health issues (e.g., pollen allergy prevalent areas, infectious disease outbreak areas, regions with high incidence of specific lifestyle diseases) from the input geographic information, and outputs optimal dissemination methods (e.g., notifications for regional medical institution collaboration, region-limited campaigns, family group notifications) and dissemination message content (e.g., pollen allergy countermeasure information, infectious disease prevention information). Examples of output include labels such as “regional medical institution collaboration notification recommended”, “pollen allergy countermeasure information prioritized”, “infectious disease prevention information prioritized”, and display/non-display flags for each item. Based on these outputs, the dissemination unit automatically switches dissemination methods and message content according to regional characteristics. Unlike conventional uniform dissemination method suggestions, AI processing is characterized by analyzing geographic information in a high-dimensional feature space and optimizing dissemination methods and content using non-conventional rules. For training the AI model, labeled data of geographic information and dissemination effectiveness or family usage rates are used, and weights are optimized using cross-entropy loss or MSE loss. This enables improvements in dissemination accuracy, promotion of service adoption according to regional characteristics, efficient data management, optimization of communication load, and overall improvement of computer technology. Application fields include regional dissemination of remote medical services, region-limited functions of health management apps, and promotion of family collaboration during infectious disease outbreaks.

The dissemination unit can analyze the social media activity of the parent generation at the time of dissemination and propose dissemination messages. For example, the dissemination unit proposes optimal dissemination messages based on the social media activity of the parent generation. The dissemination unit can also provide effective dissemination messages by analyzing the social media activity of the parent generation, and customize the content of dissemination messages by considering the social media activity of the parent generation. By analyzing the social media activity of the parent generation and proposing dissemination messages, effective messages can be provided. Some or all of the above-described processing in the dissemination unit may be performed using AI, or may be performed without using AI. For example, the dissemination unit can input social media activity data of the parent generation into a generative AI and have the generative AI execute message proposal. Specifically, the dissemination unit inputs the text of posts from the past month (up to 500 characters per post, up to 100 posts, 100×500 dimensional string array), time series data of post timestamps, and accompanying image data (one image per post, 224×224×3 dimensional RGB image tensor) into the AI. For text analysis, a Transformer-based large language model is used; for image analysis, a CNN or Vision Transformer is used; for time series analysis, an LSTM or time series encoder is used, combined in a multimodal neural network. The AI extracts keywords related to health condition (e.g., “feeling unwell”, “headache”, “lack of exercise”) and lifestyle habits (e.g., “staying up late”, “eating out”, “exercise”) from the input post text, determines dietary content and exercise status from images, and estimates lifestyle rhythm tendencies (e.g., frequent late-night posts, morning type) from post times. The AI integrates these features and outputs relevance scores for each dissemination message item (e.g., sleep habit information 0.85, dietary content information 0.80, exercise habit information 0.75), as well as input completion labels (e.g., “tendency toward lack of sleep”, “tendency toward lack of exercise”). Examples of output include labels such as “sleep habit information prioritized”, “automatic completion of dietary content”, “recommendation to input exercise habits”, and display/non-display flags for each item. Based on these outputs, the dissemination unit displays highly relevant items at the top of dissemination messages, hides unnecessary items, or enables a function to automatically supplement dissemination messages from post content. Unlike conventional simple post viewing or keyword search by humans, AI processing is characterized by autonomous execution of semantic analysis in a high-dimensional feature space, integrated feature extraction from images, text, and time series, and non-conventional rule-based branching. For training the AI model, labeled datasets of social media activity and dissemination message content selection history are used, and weights are optimized using cross-entropy loss or MSE loss. Data augmentation such as paraphrase generation for post text and noise addition to images can also be utilized. This enables improvements in the accuracy of dissemination message content, increased health awareness at the family level, efficient data management, optimization of communication load, and overall improvement of computer technology. Application fields include family dissemination of remote medical services, introduction functions of health management apps, and promotion of family collaboration in lifestyle disease prevention.

The dissemination unit can estimate a user's emotion and determine the priority of dissemination messages based on the estimated emotion. For example, when the user is nervous, the dissemination unit preferentially provides important dissemination messages. When the user is relaxed, the dissemination unit can preferentially provide detailed dissemination messages. When the user is in a hurry, the dissemination unit can preferentially provide concise dissemination messages. By determining the priority of dissemination messages according to the user's emotion, important messages can be provided preferentially. Emotion estimation is realized using, for example, an emotion engine or a generative AI with emotion estimation functionality. The generative AI may be a text generation AI (such as an LLM) or a multimodal generative AI, but is not limited to these examples. Some or all of the above-described processing in the dissemination unit may be performed using AI, or may be performed without using AI. For example, the dissemination unit can input user emotion data into a generative AI and have the generative AI execute emotion estimation. Specifically, the dissemination unit inputs voice data obtained from the user terminal (e.g., 30 seconds of speech audio, 16 kHz sampling, 1×480,000 dimensional waveform vector), facial expression images (e.g., one face image, 224×224×3 dimensional RGB tensor), and text input (e.g., speech text such as “I want to recommend medical care to my family”) into an emotion estimation AI. For voice data, a speech emotion recognition model combining convolutional neural networks and recurrent neural networks is applied; for image data, a facial expression recognition model based on ResNet or Vision Transformer is applied; and for text data, a large language model is applied. The dissemination unit receives emotion labels (e.g., “nervous”, “relaxed”, “in a hurry”) and emotion scores (e.g., nervousness 0.80, relaxation 0.10) as output from the AI. Examples of output include “nervous: 0.75, relaxed: 0.15”, “in a hurry: 0.60, normal: 0.30”, etc. The dissemination unit performs threshold judgment on these output values: if nervousness is high, important messages such as “Your family can also receive medical care” are displayed at the top; if relaxation is high, detailed explanations and additional information are preferentially displayed; if in a hurry, concise messages focusing only on key points are preferentially displayed. Unlike conventional uniform priority settings, AI processing is characterized by analyzing emotion data in a high-dimensional feature space and optimizing priority using non-conventional rules. For training the AI model, labeled data of emotional states and dissemination message priority history are used, and weights are optimized using cross-entropy loss. This enables improvements in the efficiency of important information transmission, increased user satisfaction, reduced input dropout rates, efficient data management, and overall improvement of computer technology. Application fields include family dissemination of remote medical services, introduction functions of health management apps, and promotion of online medical care for seniors.

The system according to the embodiment is not limited to the above examples, and various modifications are possible as described below. Specifically, the system allows for diverse variations in the configuration and cooperation methods of each module such as the reception unit, analysis unit, provision unit, and dissemination unit, as well as the types of AI models, data flow, input/output specifications, user interface, communication methods, and database structure. For example, the reception unit can expand the types of input data obtained from user terminals to accept various multimodal data such as voice, images, text, vital sensor data, and biometric information from wearable devices. The analysis unit can realize more advanced diagnosis prediction and risk assessment by combining Transformer-based large language models with graph neural networks, time series analysis models, autoencoders for anomaly detection, and reinforcement learning models. Examples of AI input include a 1×N dimensional user attribute vector, an M×N dimensional medical history tensor, a 224×224×3 dimensional face image tensor, a 1×480,000 dimensional voice waveform vector, and a 7×5 dimensional health condition time series vector. Examples of AI output include diagnosis labels (e.g., “suspected heart failure”, “suspected infection”), probability distributions (scores for each diagnosis candidate), recommended treatment methods (structured data), risk scores (continuous values from 0 to 1), anomaly detection flags (0/1), and explanatory texts generated by natural language generation. Subsequent processing includes threshold judgment based on output values, branching, dynamic switching of user interfaces, optimization of notification timing, and automatic message transmission to family terminals. Furthermore, the provision unit and dissemination unit dynamically control parameters of the natural language generation module (style, length, level of detail, method of expression) to realize personalized information provision according to the user's emotion and situation. For training the AI model, loss functions such as cross-entropy, MSE, triplet loss, and reinforcement learning reward functions are used, and data augmentation such as synthetic data generation, noise addition, and paraphrase generation can also be utilized. These various configuration changes and variations enable improvements in processing speed, diagnostic accuracy, user experience optimization, efficient data management, reduction of communication load, and enhancement of security, thereby realizing overall improvement of computer technology. Application fields include remote medical care, health management, nursing care support, chronic disease management, emergency medical care, lifestyle disease prevention, senior services, and family-linked health promotion services, covering a wide range of use cases.

The reception unit can customize the method of inputting medical information based on the user's hobbies and interests. For example, if the user is interested in sports, health information related to sports is preferentially input. The reception unit can also propose relaxation methods related to music if the user is interested in music. Furthermore, if the user likes traveling, the reception unit can provide information on health management during travel. By customizing the method of inputting medical information based on the user's hobbies and interests, information that is familiar to the user can be provided. Specifically, the reception unit inputs hobby and interest data obtained from the user terminal (e.g., category values such as sports, music, travel, cooking, reading, or free-text descriptions, 1×N dimensional category vectors or text strings) into the AI. For category classification of hobbies and interests, a large language model or category embedding encoder is used; for text analysis, a Transformer-based natural language processing model is used. The AI extracts features such as “sports enthusiast”, “music lover”, “travel lover” from the input data, and outputs relevance scores for each medical information item (e.g., exercise habit information 0.90, relaxation method information 0.80, travel health management information 0.85), as well as recommended input interface labels (e.g., “exercise habit input prioritized”, “relaxation method proposal”, “travel health management information display”). Examples of output include labels such as “exercise habit input screen display”, “music relaxation method proposal”, “travel health management information emphasized”, and display/non-display flags for each item. Based on these outputs, the reception unit automatically switches the input screen of the user terminal according to hobbies and interests, and highlights related health information. Unlike conventional uniform input screen display or manual customization by humans, AI processing is characterized by analyzing hobby and interest data in a high-dimensional feature space and optimizing interfaces and information items using non-conventional rules. For training the AI model, labeled data of hobbies and interests and input interface selection history are used, and weights are optimized using cross-entropy loss or MSE loss. This enables improvements in user experience, reduction of input burden, increased accuracy of medical information input, efficient data management, and overall improvement of computer technology. Application fields include remote medical reception, health management apps, personalized health promotion services, and hobby-linked health support systems.

The analysis unit can estimate a user's emotion and adjust the notification method of analysis results based on the estimated emotion. For example, if the user feels anxious, the analysis unit sends a notification that gently explains the analysis results. If the user is excited, the analysis unit can send a notification providing detailed analysis results. Furthermore, if the user is calm, the analysis unit can send a notification summarizing the analysis results concisely. By adjusting the notification method of analysis results according to the user's emotion, notifications that are easy for the user to accept can be provided. Specifically, the analysis unit inputs voice data obtained from the user terminal (e.g., 30 seconds of speech audio, 16 kHz sampling, 1×480,000 dimensional waveform vector), facial expression images (e.g., one face image, 224×224×3 dimensional RGB tensor), and text input (e.g., speech text such as “I'm worried about the result”, “I'm happy”) into an emotion estimation AI. For voice data, a speech emotion recognition model combining convolutional neural networks and recurrent neural networks is used; for image data, a facial expression recognition model based on ResNet or Vision Transformer is used; for text data, a large language model is used. The AI outputs emotion labels (e.g., “anxiety”, “excitement”, “calm”) and emotion scores (e.g., anxiety 0.80, excitement 0.60) from the input data. Examples of output include “anxiety: 0.75, excitement: 0.15”, “calm: 0.60, normal: 0.30”, etc. The analysis unit performs threshold judgment on these output values: if anxiety is high, the analysis result notification is generated in a gentle expression or reassuring style; if excitement is high, notifications including detailed figures and explanations are generated; if calmness is high, notifications summarizing only the key points are generated. Unlike conventional uniform notification method suggestions, AI processing is characterized by analyzing emotion data in a high-dimensional feature space and optimizing notification methods using non-conventional rules. For training the AI model, labeled data of emotional states and notification expression history are used, and weights are optimized using cross-entropy loss. This enables improvements in notification comprehension, increased user satisfaction, reduced input dropout rates, efficient data management, and overall improvement of computer technology. Application fields include remote medical analysis result notification, health management apps, stress care services, and online medical care for seniors.

The provision unit can customize treatment methods and advice based on the user's living environment. For example, if the user lives in an urban area, the provision unit provides advice on health risks specific to urban areas. If the user lives in a rural area, the provision unit can propose health management methods specific to rural areas. Furthermore, if the user lives in a high-rise apartment, the provision unit can provide information on health management in high-rise apartments. By customizing treatment methods and advice based on the user's living environment, information that is easy for the user to put into practice can be provided. Specifically, the provision unit inputs residential environment data obtained from the user terminal (e.g., category values such as urban area, rural area, high-rise apartment, detached house, postal code, floor number, 1×N dimensional category vector or numerical data) into the AI. For category classification of residential environment, a large language model or category embedding encoder is used; for environmental feature extraction, a multimodal neural network is used. The AI extracts features such as “urban area resident”, “rural area resident”, “high-rise apartment resident” from the input data, and outputs relevance scores for each treatment method or advice item (e.g., urban area→air pollution countermeasure 0.90, rural area→health management during farm work 0.85, high-rise apartment→countermeasure for lack of exercise due to elevator use 0.80), as well as customization labels (e.g., “urban area risk countermeasure proposal”, “rural area health management proposal”, “high-rise apartment exercise proposal”). Examples of output include labels such as “urban area: air pollution countermeasure information display”, “rural area: farm work health management information display”, “high-rise apartment: countermeasure for lack of exercise information display”, and display/non-display flags for each item. Based on these outputs, the provision unit displays treatment methods and advice optimized for the living environment on the user terminal. Unlike conventional uniform advice suggestions, AI processing is characterized by analyzing residential environment data in a high-dimensional feature space and optimizing item selection and method of expression using non-conventional rules. For training the AI model, labeled data of residential environment and advice content selection history are used, and weights are optimized using cross-entropy loss. This enables improvements in the practicality of advice, increased user satisfaction, efficient data management, and overall improvement of computer technology. Application fields include remote medical advice provision, health management apps, and living environment-linked health support services.

The reception unit can estimate a user's emotion and adjust the input interface for medical information based on the estimated emotion. For example, if the user is nervous, a simple and intuitive interface is provided. If the user is relaxed, an interface that allows input of detailed information can also be provided. Furthermore, if the user is in a hurry, a concise input interface can be provided. By adjusting the input interface for medical information according to the user's emotion, an interface that is easy for the user to use can be provided. Specifically, the reception unit inputs voice data obtained from the user terminal (e.g., 30 seconds of speech audio, 16 kHz sampling, 1×480,000 dimensional waveform vector), facial expression images (e.g., one face image, 224×224×3 dimensional RGB tensor), and text input (e.g., speech text such as “I'm anxious about input”) into an emotion estimation AI. For voice data, a speech emotion recognition model combining convolutional neural networks and recurrent neural networks is used; for image data, a facial expression recognition model based on ResNet or Vision Transformer is used; for text data, a large language model is used. The AI outputs emotion labels (e.g., “nervous”, “relaxed”, “in a hurry”) and emotion scores (e.g., nervousness 0.80, relaxation 0.10) from the input data. Examples of output include “nervous: 0.75, relaxed: 0.15”, “in a hurry: 0.60, normal: 0.30”, etc. The reception unit performs threshold judgment on these output values: if nervousness is high, a simple input screen centered on buttons and choices is displayed; if relaxation is high, a multifunctional interface allowing detailed text input and image upload is displayed; if in a hurry, a simple interface displaying only the minimum required items is presented. Unlike conventional uniform interface suggestions, AI processing is characterized by analyzing emotion data in a high-dimensional feature space and optimizing the interface using non-conventional rules. For training the AI model, labeled data of emotional states and interface selection history are used, and weights are optimized using cross-entropy loss. This enables improvements in input experience, reduction of input errors and dropout rates, efficient data management, and overall improvement of computer technology. Application fields include remote medical reception, health management apps, stress care services, and online medical care for seniors.

The analysis unit can analyze a user's past medical history and evaluate the reliability of diagnosis results. For example, the reliability of diagnosis results is scored based on past medical history. The analysis unit can also provide supplementary information to improve the reliability of diagnosis results based on past medical history, and provide feedback on the reliability of diagnosis results by analyzing past medical history. By evaluating the reliability of diagnosis results based on the user's past medical history, highly reliable information can be provided to the user. Specifically, the analysis unit inputs a medical history database saved for each user (e.g., records of the past 24 medical visits, including structured data for each visit such as diagnosis details, treatment progress, revisit status, diagnostic accuracy, error history, 24×15 dimensional table) into the AI. For handling history series, LSTM or Transformer-based time series analysis models are used; for reliability evaluation, Bayesian inference networks or ensemble learning models are used. The AI extracts features such as “consistency of diagnosis results”, “revisit rate”, “past diagnostic accuracy” from the input data, and outputs reliability scores for each diagnosis result (e.g., continuous values from 0 to 1 such as 0.95, 0.80), supplementary information labels (e.g., “additional examination recommended”, “revisit recommended”, “high diagnostic accuracy”), and feedback messages (e.g., “consistent with past diagnosis”, “caution due to high revisit rate”). Examples of output include “diagnosis reliability: 0.92”, “additional examination recommended”, “consistent with past diagnosis”, and generation of reliability evaluation reports. Based on these outputs, the analysis unit displays diagnosis results with reliability scores, supplementary information, and feedback on the user terminal. Unlike conventional simple diagnosis result display, AI processing is characterized by analyzing history data in a high-dimensional feature space and optimizing reliability evaluation and supplementary information generation using non-conventional rules. For training the AI model, labeled data of medical history and diagnosis reliability are used, and weights are optimized using cross-entropy loss or MSE loss. This enables improvements in diagnostic accuracy and reliability, increased user satisfaction, enhancement of medical safety, efficient data management, and overall improvement of computer technology. Application fields include remote medical analysis, health management apps, chronic disease management services, and medical quality evaluation systems.

The provision unit can estimate a user's emotion and adjust the timing of providing treatment methods or advice based on the estimated emotion. For example, if the user feels stressed, treatment methods or advice are provided at a timing when the user can relax. If the user is relaxed, detailed treatment methods or advice can be provided immediately. Furthermore, if the user is in a hurry, concise treatment methods or advice can be provided immediately. By adjusting the timing of providing treatment methods or advice according to the user's emotion, information that is easy for the user to accept can be provided. Specifically, the provision unit inputs voice data obtained from the user terminal (e.g., 30 seconds of speech audio, 16 kHz sampling, 1×480,000 dimensional waveform vector), facial expression images (e.g., one face image, 224×224×3 dimensional RGB tensor), and text input (e.g., speech text such as “I'm busy now”, “I'm calm”) into an emotion estimation AI. For voice data, a speech emotion recognition model combining convolutional neural networks and recurrent neural networks is used; for image data, a facial expression recognition model based on ResNet or Vision Transformer is used; for text data, a large language model is used. The AI outputs emotion labels (e.g., “stress”, “relaxation”, “in a hurry”) and emotion scores (e.g., stress 0.80, relaxation 0.10) from the input data. Examples of output include “stress: 0.75, relaxation: 0.15”, “in a hurry: 0.60, normal: 0.30”, etc. The provision unit performs threshold judgment on these output values: if stress is high, notification timing is delayed; if relaxation is high, detailed advice is sent immediately; if in a hurry, only key points are sent immediately in short sentences. Unlike conventional uniform notification timing settings, AI processing is characterized by analyzing emotion data in a high-dimensional feature space and optimizing timing using non-conventional rules. For training the AI model, labeled data of emotional states and notification timing history are used, and weights are optimized using cross-entropy loss. This enables improvements in advice acceptance, increased user satisfaction, reduced input dropout rates, efficient data management, and overall improvement of computer technology. Application fields include remote medical advice provision, health management apps, stress care services, and online medical care for seniors.

The dissemination unit can analyze a user's social network and select the optimal dissemination route. For example, the dissemination unit identifies influential friends based on the user's friendship network and sends dissemination messages through those friends. The dissemination unit can also identify users with high dissemination effect based on the number of followers and send dissemination messages through those users. Furthermore, the dissemination unit analyzes the activity status of the user's social network and selects the optimal dissemination timing. By selecting the optimal dissemination route based on the user's social network, effective dissemination becomes possible. Specifically, the dissemination unit inputs social network data obtained from the user terminal (e.g., friend list, number of followers, mutual relationship graph, post history, 1×N dimensional network structure data or time series activity data) into the AI. For network structure analysis, a graph neural network is used; for activity status analysis, a time series analysis model or attention mechanism is used. The AI extracts features such as “nodes (friends/followers) with high influence”, “users with high activity frequency”, “routes with high dissemination effect” from the input data, and recommends optimal dissemination routes (e.g., via friend A, via follower B, via group chat) and dissemination timing (e.g., weekday evenings, weekend mornings). Examples of output include labels such as “dissemination via friend A recommended”, “dissemination via follower B recommended”, “dissemination on weekend morning recommended”, and recommendation scores (e.g., friend A 0.85, follower B 0.80). Based on these outputs, the dissemination unit automatically selects dissemination routes and timing and sends dissemination messages. Unlike conventional simple mass transmission or manual selection by humans, AI processing is characterized by analyzing network structure data in a high-dimensional feature space and optimizing dissemination routes and timing using non-conventional rules. For training the AI model, labeled data of network structure and dissemination effect history are used, and weights are optimized using cross-entropy loss or MSE loss. This enables improvements in dissemination efficiency, increased service adoption rate, efficient data management, optimization of communication load, and overall improvement of computer technology. Application fields include family dissemination of remote medical services, introduction functions of health management apps, and SNS-linked health promotion services.

The analysis unit can estimate a user's emotion and adjust the visualization method of analysis results based on the estimated emotion. For example, if the user feels anxious, the analysis unit provides simple and highly visible graphs. If the user is excited, the analysis unit can provide graphs containing detailed data. Furthermore, if the user is calm, the analysis unit can provide interactive graphs. By adjusting the visualization method of analysis results according to the user's emotion, analysis results that are easy for the user to understand can be provided. Specifically, the analysis unit inputs voice data obtained from the user terminal (e.g., 30 seconds of speech audio, 16 kHz sampling, 1×480,000 dimensional waveform vector), facial expression images (e.g., one face image, 224×224×3 dimensional RGB tensor), and text input (e.g., speech text such as “I want to see a graph”) into an emotion estimation AI. For voice data, a speech emotion recognition model combining convolutional neural networks and recurrent neural networks is used; for image data, a facial expression recognition model based on ResNet or Vision Transformer is used; for text data, a large language model is used. The AI outputs emotion labels (e.g., “anxiety”, “excitement”, “calm”) and emotion scores (e.g., anxiety 0.80, excitement 0.60) from the input data. Examples of output include “anxiety: 0.75, excitement: 0.15”, “calm: 0.60, normal: 0.30”, etc. The analysis unit performs threshold judgment on these output values: if anxiety is high, simple visualizations such as line graphs or bar graphs are displayed; if excitement is high, complex graphs with detailed figures and annotations are displayed; if calmness is high, interactive graphs that the user can operate are displayed. Unlike conventional uniform graph display, AI processing is characterized by analyzing emotion data in a high-dimensional feature space and optimizing visualization methods using non-conventional rules. For training the AI model, labeled data of emotional states and graph display history are used, and weights are optimized using cross-entropy loss. This enables improvements in understanding of analysis results, increased user satisfaction, efficient data management, and overall improvement of computer technology. Application fields include remote medical analysis result display, health management apps, stress care services, and online medical care for seniors.

The provision unit can customize treatment methods and advice based on the user's lifestyle. For example, if the user has a nocturnal lifestyle, the provision unit proposes health management methods that can be practiced at night. If the user has a desk work-centered lifestyle, the provision unit can propose exercises that can be done during desk work. Furthermore, if the user prefers outdoor activities, the provision unit can provide information on health management for outdoor activities. By customizing treatment methods and advice based on the user's lifestyle, information that is easy for the user to put into practice can be provided. Specifically, the provision unit inputs lifestyle data obtained from the user terminal (e.g., lifestyle rhythm, work style, hobbies, activity time zone, 1×N dimensional category vector or time series activity data) into the AI. For lifestyle classification, a large language model or category embedding encoder is used; for activity pattern extraction, a time series analysis model or clustering algorithm is used. The AI extracts features such as “nocturnal lifestyle”, “desk work-centered”, “frequent outdoor activities” from the input data, and outputs relevance scores for each treatment method or advice item (e.g., nighttime health management information 0.90, desk work exercise information 0.85, outdoor health management information 0.80), as well as customization labels (e.g., “nighttime health management proposal”, “desk work exercise proposal”, “outdoor health management proposal”). Examples of output include labels such as “nighttime health management information display”, “desk work exercise proposal”, “outdoor health management information display”, and display/non-display flags for each item. Based on these outputs, the provision unit displays treatment methods and advice optimized for the lifestyle on the user terminal. Unlike conventional uniform advice suggestions, AI processing is characterized by analyzing lifestyle data in a high-dimensional feature space and optimizing item selection and method of expression using non-conventional rules. For training the AI model, labeled data of lifestyle and advice content selection history are used, and weights are optimized using cross-entropy loss. This enables improvements in the practicality of advice, increased user satisfaction, efficient data management, and overall improvement of computer technology. Application fields include remote medical advice provision, health management apps, and lifestyle-linked health support services.

The dissemination unit can estimate a user's emotion and adjust the content of dissemination messages based on the estimated emotion. For example, if the user is nervous, a message that provides reassurance is provided. If the user is relaxed, a message containing detailed information can also be provided. Furthermore, if the user is in a hurry, a concise message focusing on key points can be provided. By adjusting the content of dissemination messages according to the user's emotion, messages that are easy for the user to accept can be provided. Specifically, the dissemination unit inputs voice data obtained from the user terminal (e.g., 30 seconds of speech audio, 16 kHz sampling, 1×480,000 dimensional waveform vector), facial expression images (e.g., one face image, 224×224×3 dimensional RGB tensor), and text input (e.g., speech text such as “I want to recommend medical care to my family”) into an emotion estimation AI. For voice data, a speech emotion recognition model combining convolutional neural networks and recurrent neural networks is applied; for image data, a facial expression recognition model based on ResNet or Vision Transformer is applied; for text data, a large language model is applied. The dissemination unit receives emotion labels (e.g., “nervous”, “relaxed”, “in a hurry”) and emotion scores (e.g., nervousness 0.80, relaxation 0.10) as output from the AI. Examples of output include “nervous: 0.75, relaxed: 0.15”, “in a hurry: 0.60, normal: 0.30”, etc. The dissemination unit performs threshold judgment on these output values: if nervousness is high, a short sentence providing reassurance such as “Your family can also receive medical care” is generated; if relaxation is high, a long message including detailed explanations and additional information is generated; if in a hurry, a concise message focusing only on key points is generated. Unlike conventional uniform message content suggestions, AI processing is characterized by analyzing emotion data in a high-dimensional feature space and optimizing content using non-conventional rules. For training the AI model, labeled data of emotional states and dissemination message content history are used, and weights are optimized using cross-entropy loss. This enables improvements in the efficiency of important information transmission, increased user satisfaction, reduced input dropout rates, efficient data management, and overall improvement of computer technology. Application fields include family dissemination of remote medical services, introduction functions of health management apps, and promotion of online medical care for seniors.

Below, the processing flow of Example of the Embodiment is briefly described. Specifically, the system is configured such that each module—the reception unit, analysis unit, provision unit, and dissemination unit—operate in cooperation. The system receives input data sent from user terminals (smartphones, tablets, PCs, etc.) via the reception unit, and preprocesses parameters such as age (integer value), gender (category value), past medical history (text or coded data), and current symptoms (text or selection) as a 1×N dimensional numerical array. The reception unit performs preprocessing such as missing value completion, one-hot encoding of category values, and tokenization of text on these data and transfers them to the analysis unit. The analysis unit uses, for example, a Transformer-based large language model or a multimodal neural network capable of handling mixed text and numerical inputs, with the user information vector and past medical history tensor (M×N dimensions, where M is the number of history records) as input. Examples of input include: 70-year-old male with a history of hypertension and “recent shortness of breath”; 85-year-old female with a history of diabetes and “loss of appetite”; 65-year-old male with no past medical history and “fever”. The analysis unit searches for similar cases in a high-dimensional space from a database of past medical cases (on the order of hundreds of thousands of cases), extracts user-specific features using a feature extraction layer (multi-layer perceptron or attention mechanism), and generates as output diagnosis labels (e.g., “suspected heart failure”, “suspected infection”), probability distributions (confidence scores for each diagnosis candidate), and recommended treatment methods (structured data such as drug names, lifestyle guidance). Examples of output include probability distributions such as “suspected heart failure: 0.82, suspected infection: 0.12”, treatment proposals such as “continuation of antihypertensive drugs, guidance on salt restriction”, and advice such as “additional examination recommended”. The provision unit receives these outputs and generates and displays individualized treatment methods and advice on the user terminal using a natural language generation module. The dissemination unit automatically generates and sends messages such as “Would your family also like to use online medical care?” to the child generation's terminal based on the history and usage status when the parent generation receives medical care. Unlike conventional human physician interviews, diagnosis, and information transmission to families, these processes are characterized by the AI autonomously executing similarity calculations in high-dimensional feature space, rule-based branching, probabilistic inference, and natural language generation using non-conventional algorithms. For training the AI model, cross-entropy or MSE is used as the loss function, and weight optimization is performed by gradient descent. Data augmentation such as synthetic generation of case data and noise addition can also be utilized. This enables improvements in diagnostic accuracy, reduction of input burden, promotion of service adoption at the family level, efficient data management, optimization of communication load, and overall improvement of computer technology. Application fields include remote medical care, home care, chronic disease management, nursing care support, and health promotion services, and are particularly beneficial for improving medical access for the elderly generation and at the family level.

Step 1: The reception unit receives as input a small number of parameters such as the user's basic information and past medical history. The user's basic information includes, for example, age, gender, medical history, and current symptoms. Step 2: The analysis unit analyzes the information received by the reception unit and supplements information necessary for diagnosis using unique information of each individual as a key. The analysis unit predicts the user's symptoms based on past medical data and similar case data. Step 3: The provision unit provides appropriate treatment methods or advice based on the diagnosis result predicted by the analysis unit. The provision unit proposes, for example, prescriptions for medication or improvements to lifestyle habits. Step 4: The dissemination unit sends a message prompting the child generation to use a similar service when the parent generation receives online medical care. This promotes the use of online medical care by the entire family. Specifically, in Step 1, the reception unit receives parameters such as age (integer value), gender (categorical value), medical history (text or coded data), and current symptoms (text or selectable options) sent from the user terminal as a 1×N dimensional numerical array, and performs preprocessing such as missing value imputation, one-hot encoding of categorical values, and tokenization of text. In Step 2, the analysis unit uses Transformer-based large language models and multimodal neural networks, taking as input the user information vector and past medical history tensor (M×N dimensions), searches for similar cases in the past medical database in a high-dimensional space, and extracts user-specific features in the feature extraction layer. In the diagnosis prediction layer, it generates diagnosis labels (e.g., “suspected heart failure,” “suspected infection,” etc.), probability distributions (scores for each diagnosis candidate), and recommended treatment methods (structured data). Examples of output include probability distributions such as “suspected heart failure: 0.82, suspected infection: 0.12,” treatment proposals such as “continuation of antihypertensive medication, guidance on salt restriction,” and advice such as “recommend additional tests.” In Step 3, the provision unit receives these outputs and generates and displays personalized treatment methods or advice to the user terminal using a natural language generation module. In Step 4, the dissemination unit automatically generates and sends messages such as “Would your family also like to use online medical care?” to the child generation's terminal based on the history and usage status of the parent generation's medical care. These processes are technically characterized by the fact that, unlike conventional human physician-based interviews, diagnoses, and information transmission to families, AI autonomously executes non-conventional algorithms such as similarity calculation in high-dimensional feature space, rule-based branching, probabilistic inference, and natural language generation. For training the AI model, loss functions such as cross-entropy and MSE are used, and weight optimization is performed by gradient descent. Data augmentation, such as synthetic generation of case data and addition of noise, can also be utilized. As a result, improvements in computer technology itself are achieved, including enhanced diagnostic accuracy, reduced input burden, promotion of service dissemination at the family level, efficient data management, and optimization of communication load. Application fields include telemedicine, home care, chronic disease management, nursing care support, and health promotion services, contributing particularly to improved medical access for the elderly generation and at the family level.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned reception unit, analysis unit, provision unit, and dissemination unit is implemented by at least one of, for example, the smart device 14 and the data processing apparatus 12. For example, the reception unit is implemented by a control unit 46A of the smart device 14 and receives as input a small number of parameters such as the user's basic information or past medical history. The analysis unit is implemented, for example, by a specific processing unit 290 of the data processing apparatus 12, analyzes the information received by the reception unit using generative AI, and supplements information necessary for diagnosis. The provision unit is implemented, for example, by the control unit 46A of the smart device 14 and provides an appropriate treatment method or advice based on a diagnosis result predicted by the analysis unit. The dissemination unit is implemented, for example, by the specific processing unit 290 of the data processing apparatus 12 and sends a message prompting the child generation to use a similar service when the parent generation receives online medical care. The correspondence between each unit and the apparatus or control unit is not limited to the examples described above and various modifications are possible.

### Second Embodiment

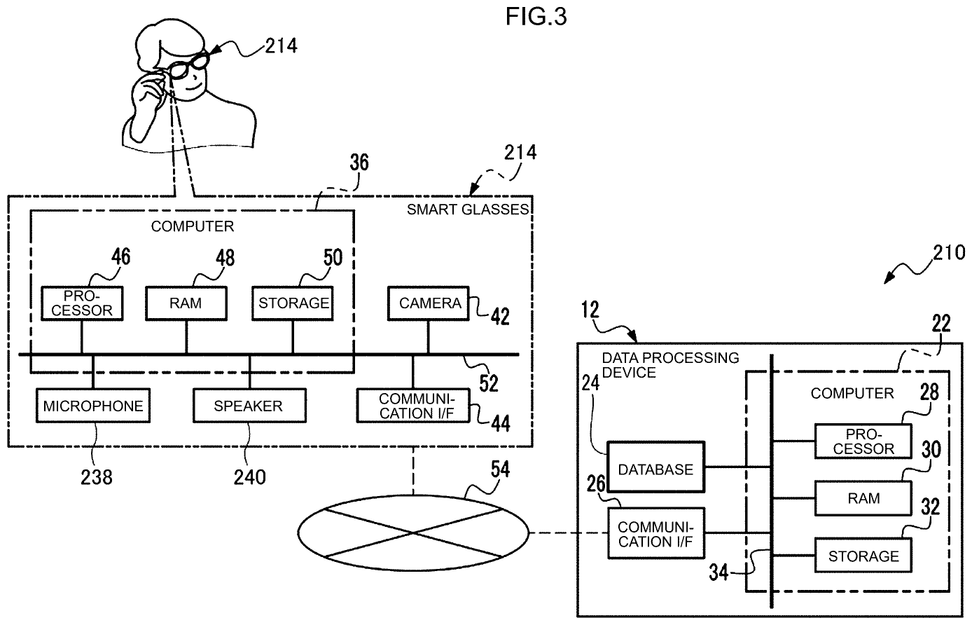

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

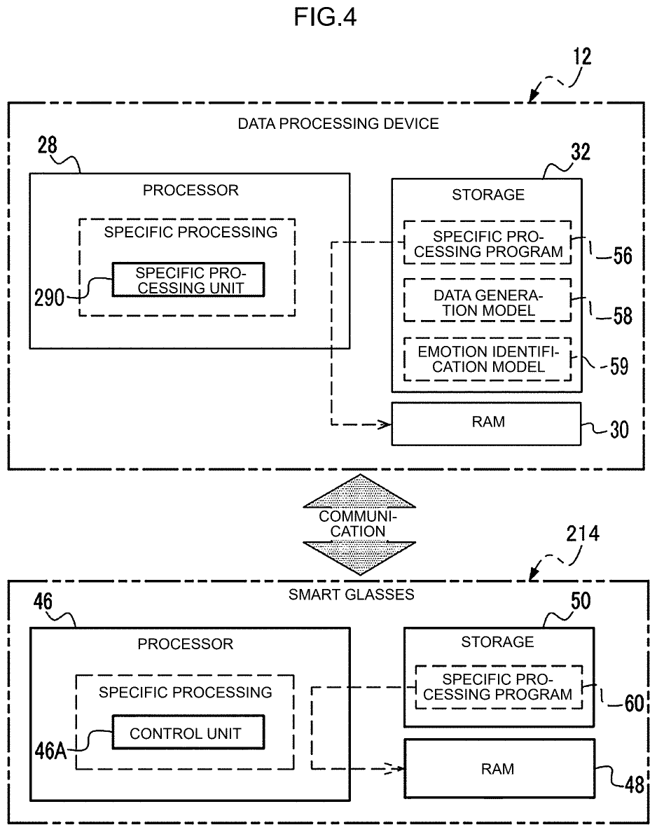

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned reception unit, analysis unit, provision unit, and dissemination unit is implemented by at least one of, for example, the smart glasses 214 and the data processing apparatus 12. For example, the reception unit is implemented by a control unit 46A of the smart glasses 214 and receives as input a small number of parameters such as the user's basic information or past medical history. The analysis unit is implemented, for example, by a specific processing unit 290 of the data processing apparatus 12, analyzes the information received by the reception unit using generative AI, and supplements information necessary for diagnosis. The provision unit is implemented, for example, by the control unit 46A of the smart glasses 214 and provides an appropriate treatment method or advice based on a diagnosis result predicted by the analysis unit. The dissemination unit is implemented, for example, by the specific processing unit 290 of the data processing apparatus 12 and sends a message prompting the child generation to use a similar service when the parent generation receives online medical care. The correspondence between each unit and the apparatus or control unit is not limited to the examples described above and various modifications are possible.

### Third Embodiment

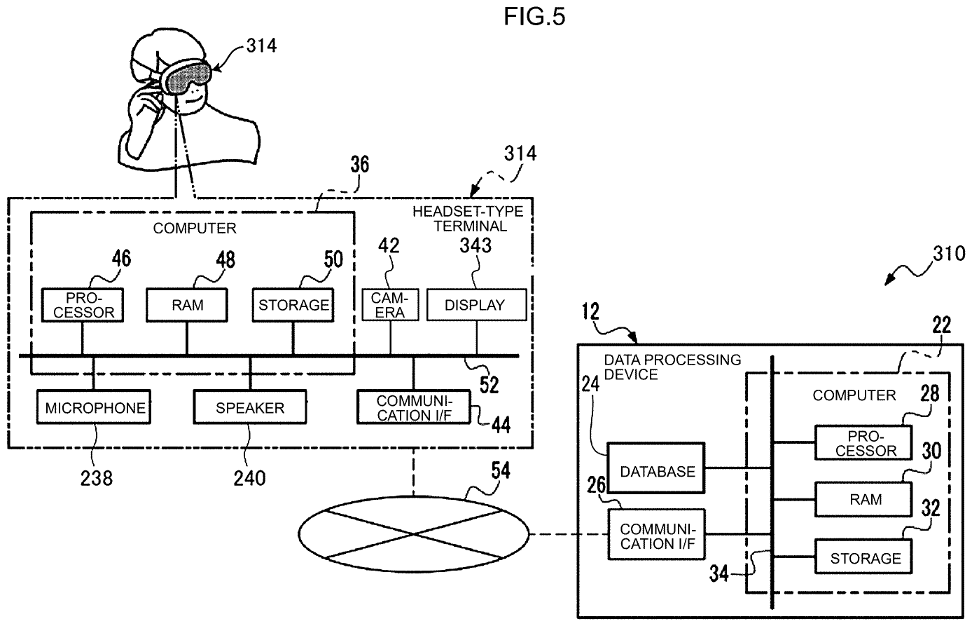

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

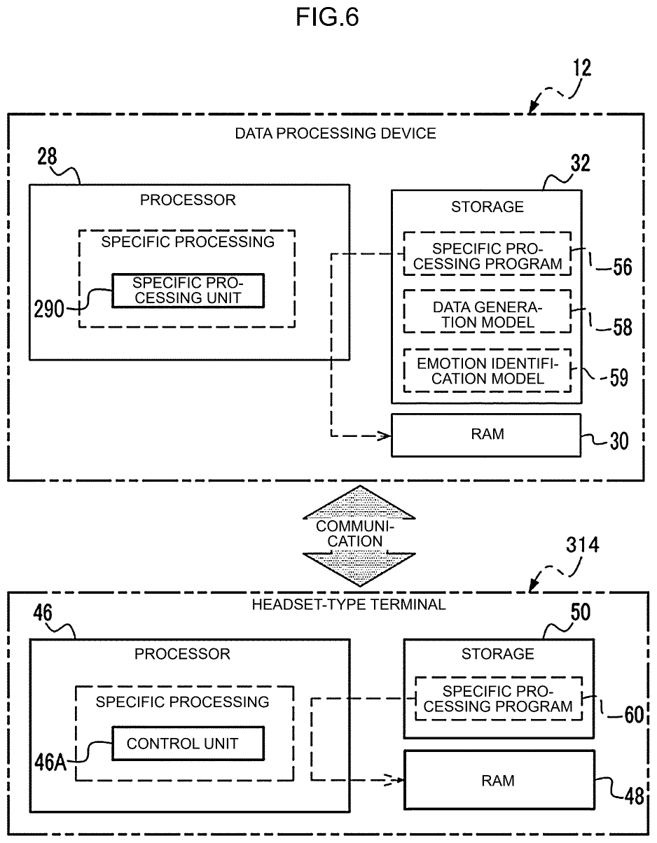

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned reception unit, analysis unit, provision unit, and dissemination unit is implemented by at least one of, for example, the headset-type terminal 314 and the data processing apparatus 12. For example, the reception unit is implemented by a control unit 46A of the headset-type terminal 314 and receives as input a small number of parameters such as the user's basic information or past medical history. The analysis unit is implemented, for example, by a specific processing unit 290 of the data processing apparatus 12, analyzes the information received by the reception unit using generative AI, and supplements information necessary for diagnosis. The provision unit is implemented, for example, by the control unit 46A of the headset-type terminal 314 and provides an appropriate treatment method or advice based on a diagnosis result predicted by the analysis unit. The dissemination unit is implemented, for example, by the specific processing unit 290 of the data processing apparatus 12 and sends a message prompting the child generation to use a similar service when the parent generation receives online medical care. The correspondence between each unit and the apparatus or control unit is not limited to the examples described above and various modifications are possible.

### Fourth Embodiment

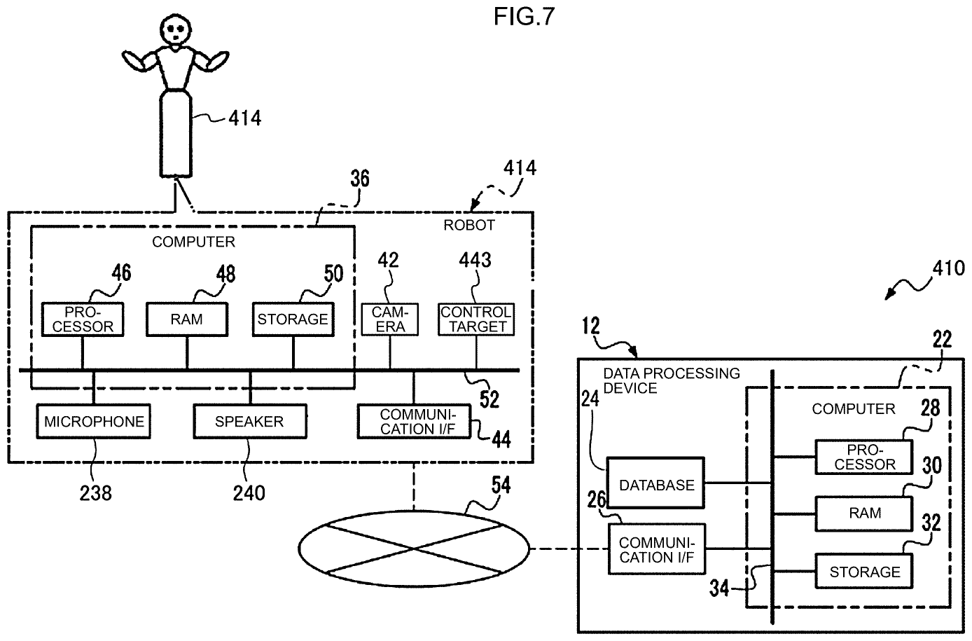

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

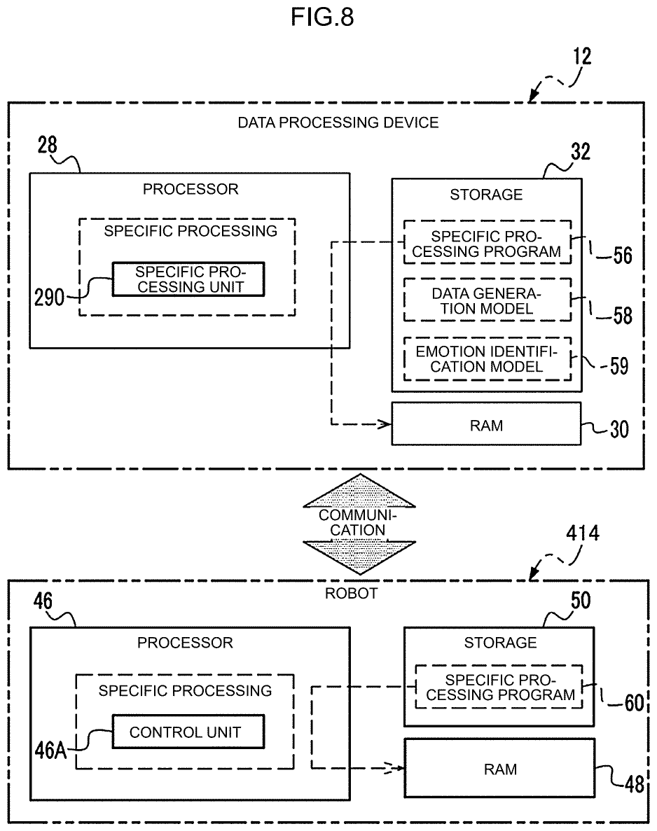

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned reception unit, analysis unit, provision unit, and dissemination unit is implemented by at least one of, for example, the robot 414 and the data processing apparatus 12. For example, the reception unit is implemented by a control unit 46A of the robot 414 and receives as input a small number of parameters such as the user's basic information or past medical history. The analysis unit is implemented, for example, by a specific processing unit 290 of the data processing apparatus 12, analyzes the information received by the reception unit using generative AI, and supplements information necessary for diagnosis. The provision unit is implemented, for example, by the control unit 46A of the robot 414 and provides an appropriate treatment method or advice based on a diagnosis result predicted by the analysis unit. The dissemination unit is implemented, for example, by the specific processing unit 290 of the data processing apparatus 12 and sends a message prompting the child generation to use a similar service when the parent generation receives online medical care. The correspondence between each unit and the apparatus or control unit is not limited to the examples described above and various modifications are possible.

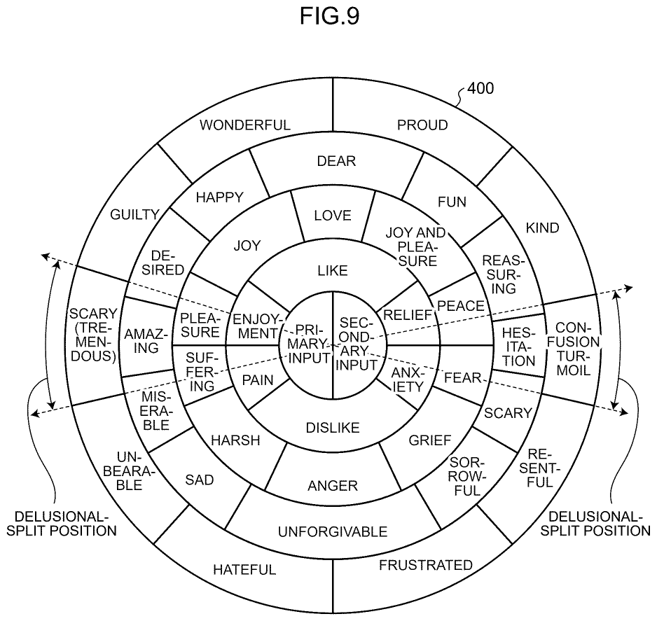

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

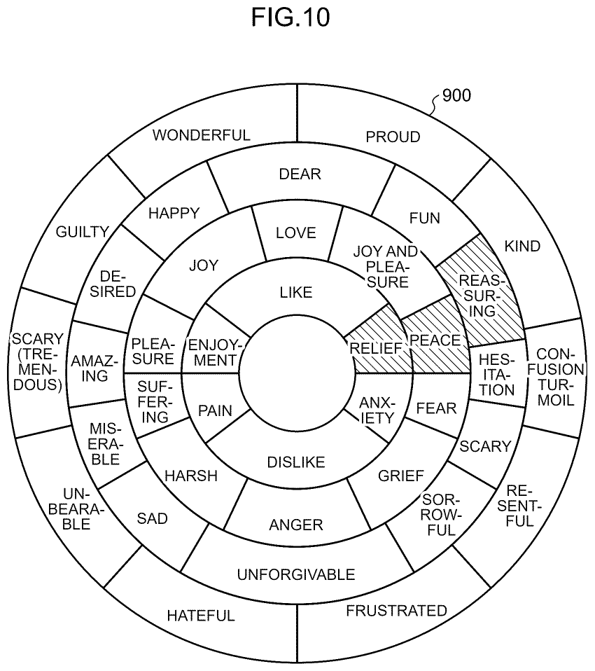

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a reception unit configured to receive as input a small number of parameters of a user's basic information or past medical history; an analysis unit configured to analyze the information received by the reception unit and supplement information necessary for diagnosis using unique information of each individual as a key; a provision unit configured to provide a treatment method or advice based on a diagnosis result predicted by the analysis unit; and a dissemination unit configured to send a message prompting the child generation to use a similar service when the parent generation receives online medical care.
(Supplementary Note 2) The system according to Supplementary Note 1, wherein the reception unit is configured to estimate a user's emotion and adjust the timing of inputting medical information based on the estimated emotion of the user.
(Supplementary Note 3) The system according to Supplementary Note 1, wherein the reception unit is configured to analyze a user's past medical history and select an appropriate input method.
(Supplementary Note 4) The system according to Supplementary Note 1, wherein the reception unit is configured to perform filtering based on the user's current health condition or lifestyle habits when inputting medical information.
(Supplementary Note 5) The system according to Supplementary Note 1, wherein the reception unit is configured to estimate a user's emotion and determine the priority of medical information to be input based on the estimated emotion of the user.
(Supplementary Note 6) The system according to Supplementary Note 1, wherein the reception unit is configured to preferentially input highly relevant information based on the user's geographic location information when inputting medical information.
(Supplementary Note 7) The system according to Supplementary Note 1, wherein the reception unit is configured to analyze a user's social media activity and input relevant information when inputting medical information.
(Supplementary Note 8) The system according to Supplementary Note 1, wherein the analysis unit is configured to estimate a user's emotion and adjust the method of presenting analysis based on the estimated emotion of the user.
(Supplementary Note 9) The system according to Supplementary Note 1, wherein the analysis unit is configured to adjust the level of detail of analysis based on the importance of medical information during analysis.
(Supplementary Note 10) The system according to Supplementary Note 1, wherein the analysis unit is configured to apply different analysis algorithms according to the category of medical information during analysis.
(Supplementary Note 11) The system according to Supplementary Note 1, wherein the analysis unit is configured to estimate a user's emotion and adjust the length of analysis based on the estimated emotion of the user.
(Supplementary Note 12) The system according to Supplementary Note 1, wherein the analysis unit is configured to determine the priority of analysis based on the timing of submission of medical information during analysis.
(Supplementary Note 13) The system according to Supplementary Note 1, wherein the analysis unit is configured to adjust the order of analysis based on the relevance of medical information during analysis.
(Supplementary Note 14) The system according to Supplementary Note 1, wherein the provision unit is configured to estimate a user's emotion and adjust the method of presenting a treatment method or advice to be provided based on the estimated emotion of the user.
(Supplementary Note 15) The system according to Supplementary Note 1, wherein the provision unit is configured to adjust the level of detail of provision based on the importance of the diagnosis result during provision.
(Supplementary Note 16) The system according to Supplementary Note 1, wherein the provision unit is configured to apply different provision algorithms according to the category of the diagnosis result during provision.
(Supplementary Note 17) The system according to Supplementary Note 1, wherein the provision unit is configured to estimate a user's emotion and adjust the length of a treatment method or advice to be provided based on the estimated emotion of the user.
(Supplementary Note 18) The system according to Supplementary Note 1, wherein the provision unit is configured to determine the priority of provision based on the timing of submission of the diagnosis result during provision.
(Supplementary Note 19) The system according to Supplementary Note 1, wherein the provision unit is configured to determine the priority of provision based on the timing of submission of the diagnosis result during provision.
(Supplementary Note 20) The system according to Supplementary Note 1, wherein the provision unit is configured to adjust the order of provision based on the relevance of the diagnosis result during provision.
(Supplementary Note 21) The system according to Supplementary Note 1, wherein the dissemination unit is configured to estimate a user's emotion and adjust the method of presenting a dissemination message based on the estimated emotion of the user.
(Supplementary Note 22) The system according to Supplementary Note 1, wherein the dissemination unit is configured to analyze the medical history of the parent generation during dissemination and select an optimal dissemination method.
(Supplementary Note 23) The system according to Supplementary Note 1, wherein the dissemination unit is configured to customize a dissemination message based on the current health condition of the parent generation during dissemination.
(Supplementary Note 24) The system according to Supplementary Note 1, wherein the dissemination unit is configured to estimate a user's emotion and determine the priority of dissemination messages based on the estimated emotion of the user.
(Supplementary Note 25) The system according to Supplementary Note 1, wherein the dissemination unit is configured to select an optimal dissemination method in consideration of the geographic location information of the parent generation during dissemination.
(Supplementary Note 26) The system according to Supplementary Note 1, wherein the dissemination unit is configured to analyze the social media activity of the parent generation during dissemination and propose a dissemination message.

What is claimed is:

1. A system comprising:
circuitry configured to:
receive, from a client terminal via a communication interface and a packet-switched network, structured data comprising at least one of text data, voice data, or image data;
store the received structured data in a database;
analyze the structured data stored in the database by inputting the structured data into a data generation model obtained by deep learning on a neural network to generate inference data;
estimate an emotion of a user by applying an emotion identification model to sensor data received from the client terminal via the communication interface;
adjust at least one of a format or a level of detail of the inference data based on the estimated emotion; and
transmit the inference data to the client terminal via the communication interface and the packet-switched network, the inference data causing the client terminal to present the inference data to the user.

2. The system according to claim 1, wherein the structured data comprises user attribute data and historical data associated with the user, the user attribute data comprising at least one of age, gender, or current status information.

3. The system according to claim 1, wherein the circuitry is further configured to preprocess the structured data by performing at least one of missing value imputation, one-hot encoding of categorical values, or tokenization of text data to generate a multidimensional numerical array.

4. The system according to claim 1, wherein the data generation model comprises a Transformer-based large language model or a multimodal neural network, and wherein the circuitry is configured to search for similar records in the database in a high-dimensional feature space and extract user-specific features using a feature extraction layer comprising at least one of a multi-layer perceptron or an attention mechanism.

5. The system according to claim 1, wherein the inference data comprises at least one of a classification label, a probability distribution over a plurality of categories, or a recommendation text generated by a natural language generation module.

6. The system according to claim 1, wherein the circuitry is further configured to adjust a timing of receiving the structured data from the client terminal based on the estimated emotion, such that when the estimated emotion indicates stress, the circuitry delays the receiving, and when the estimated emotion indicates relaxation, the circuitry receives the structured data immediately.

7. The system according to claim 1, wherein the circuitry is further configured to analyze a past reception history stored in the database and select an optimal data reception method using at least one of an LSTM model or a Transformer-based time series analysis model.

8. The system according to claim 1, wherein the circuitry is further configured to perform filtering on the structured data based on attribute information of the user comprising at least one of a current condition or a lifestyle habit, and to input only filtered structured data satisfying relevance criteria into the data generation model.

9. The system according to claim 1, wherein the circuitry is further configured to determine a priority of the structured data to be received based on the estimated emotion, such that when the estimated emotion indicates stress, the circuitry prioritizes structured data having a high importance attribute.

10. The system according to claim 1, wherein the circuitry is further configured to receive geographic location information of the user from the client terminal via the communication interface, and to preferentially receive structured data associated with a geographic region corresponding to the geographic location information.

11. The system according to claim 1, wherein the circuitry is further configured to receive social media activity data of the user from the client terminal, analyze the social media activity data using a natural language processing model to extract keywords, and select structured data to receive based on the extracted keywords.

12. The system according to claim 1, wherein the circuitry is further configured to adjust a level of detail of the analysis based on an importance score associated with the structured data, such that for structured data having a high importance score, the circuitry performs detailed analysis, and for structured data having a low importance score, the circuitry performs concise analysis.

13. The system according to claim 1, wherein the circuitry is further configured to apply different analysis algorithms according to a category of the structured data.

14. The system according to claim 1, wherein the circuitry is further configured to adjust a length of the inference data based on the estimated emotion, such that when the estimated emotion indicates stress, the circuitry generates concise inference data, and when the estimated emotion indicates relaxation, the circuitry generates detailed inference data.

15. The system according to claim 1, wherein the circuitry is further configured to determine a priority of generating the inference data based on a submission timing associated with the structured data, such that structured data having a more recent submission timing is analyzed with a higher priority.

16. The system according to claim 1, wherein the circuitry is further configured to transmit, to a second client terminal associated with a related user via the communication interface and the packet-switched network, a dissemination message generated based on a usage status of the user, the dissemination message prompting the related user to use a similar service.

17. The system according to claim 16, wherein the circuitry is further configured to analyze a historical record of the user stored in the database and select an optimal dissemination method for the dissemination message, the dissemination method comprising at least one of an email notification, a push notification, or an in-app banner.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model;
a database; and
circuitry configured to:
receive, from the client terminal via the communication interface, structured data comprising at least one of text data input via the touch panel, voice data captured by the microphone, or image data captured by the camera;
store the received structured data in the database;
analyze the structured data stored in the database by inputting the structured data into the data generation model to generate inference data comprising at least one of a classification label, a probability score, or a recommendation text;
estimate an emotion of a user by applying the emotion identification model to at least one of the voice data captured by the microphone or the image data captured by the camera;
adjust at least one of a format, a level of detail, or a length of the inference data based on the estimated emotion; and
transmit the inference data to the client terminal via the communication interface and the packet-switched network, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface, a memory storing a data generation model obtained by deep learning on a neural network and an emotion identification model, and a database, the method comprising:
receiving, from a client terminal via the communication interface and a packet-switched network, structured data comprising at least one of text data, voice data, or image data;
storing the received structured data in the database;
analyzing the structured data stored in the database by inputting the structured data into the data generation model to generate inference data;
estimating an emotion of a user by applying the emotion identification model to sensor data received from the client terminal via the communication interface;
adjusting at least one of a format or a level of detail of the inference data based on the estimated emotion; and
transmitting the inference data to the client terminal via the communication interface and the packet-switched network, the inference data causing the client terminal to present the inference data to the user.
